(19) United States
(12) Patent Application Publication
(10) Pub. No.: US 2003/0088245 A1
(43) Pub. Date: May 8, 2003

(54) Methods and apparatus for electrosurgical ventriculostomy

(75) Inventors: Jean Woloszko, Mountain View, CA (US); David C. Hovda, Mountain View, CA (US)

Correspondence Address: ARTHROCARE CORPORATION, 680 VAQUEROS AVENUE, SUNNYVALE, CA, 94085-3523, US

(73) Assignee: ArthroCare Corporation, Sunnyvale, CA

(21) Appl. No.: 10/288,227

(22) Filed: Nov. 4, 2002

Related U.S. Application Data
(60) Provisional application No. 60/350,293, filed on Nov. 2, 2001.

(51) Int. Cl. A61B 18/14

(52) U.S. Cl. 606/41

(57) ABSTRACT

Methods and apparatus for electrosurgical treatment of hydrocephalus. A method of the invention comprises electrosurgical fenestration of the floor of the third ventricle using an electrosurgical probe or catheter. The probe or catheter may be introduced via an access hole in the patient's cranium. The access hole can be formed mechanically or electrosurgically. A stoma or window in the third ventricle can be enlarged electrosurgically and/or tissue surrounding the stoma can be coagulated, in order to maintain patency of the stoma. According to another aspect of the invention, a method of establishing patency in an occluded cerebral aqueduct comprises guiding an electrosurgical catheter into the cerebral aqueduct, positioning an active electrode in at least close proximity to the occlusion, and applying an ablative voltage to the active electrode to form a channel within the cerebral aqueduct.

### RELATED APPLICATION

This application is a non-provisional of U.S. Provisional application No. 60/350,293 filed Nov. 2, 2001, which is incorporated by reference.

### BACKGROUND OF THE INVENTION

The present invention relates generally to the field of electrosurgery, and more particularly to surgical devices and methods which employ high frequency electrical energy to ablate, fenestrate, channel, shrink, coagulate, or otherwise modify a target tissue. The present invention is particularly suited for the treatment of tissue in or around the central nervous system. The present invention further relates to the treatment of hydrocephalus, and to methods for performing a third ventriculostomy.

Hydrocephalus is a common condition characterized by an excessive accumulation of cerebrospinal fluid (CSF) in the cerebral ventricles, resulting in dilation of the ventricles and elevated intracranial pressure. If untreated, hydrocephalus may result in excessive enlargement of the cranium and atrophy of the brain. The cerebral ventricles comprise the right and left lateral ventricles, the third ventricle, and the fourth ventricle. Each of the right and left lateral ventricles includes an anterior horn, an occipital horn and a temporal horn. The third ventricle lies generally between the anterior horn and the inferior horn of the right and left lateral ventricles. The fourth ventricle lies inferior to both the third ventricle and the temporal horn of the right and left lateral ventricles. Each of the right and left lateral ventricles is in communication with the third ventricle via the interventricular foramen of Monro. The third ventricle is, in turn, in communication with the fourth ventricle via the cerebral aqueduct. (See, for example, F. H. Netter, M.D., Atlas of Human Anatomy, 2^{nd} Edition, 3^{rd} Printing, Novartis, East Hanover, N.J., 1999.)

Hydrocephalus may be congenital, or may result from trauma, tumors, postsub-arachnoid hemorrhage, or post-meningitis. Treatment of hydrocephalus is one of the most common neurosurgical procedures. There are two types of hydrocephalus: obstructive hydrocephalus and communicating hydrocephalus. Obstructive hydrocephalus is caused by a block to drainage of CSF from the ventricles, most commonly due to occlusion of the cerebral aqueduct (between the third and fourth ventricles). Thus, obstructive hydrocephalus is due to lack of drainage of fluid from the ventricles. In contrast, in communicating hydrocephalus, CSF drains from the brain, but then accumulates due to a defect in absorption of CSF.

One conventional procedure for the treatment of obstructive hydrocephalus is a shunt procedure which involves introducing a catheter into one of the lateral ventricles to drain excess CSF, via a valve and tube, into the venous or peritoneal spaces (e.g., a ventriculo-peritoneal (VP) shunt procedure). Techniques for VP shunt procedures were developed in the 1950's, and the procedure has been practiced for more than 40 years. However, shunt procedures for treatment of hydrocephalus have a number of drawbacks. For example, VP shunts often need to be revised, for example due to obstruction of the catheter/valve/tubing, infection, and, in the case of children, growth of the patient. Furthermore, antibiotics are frequently prescribed for patients having shunts in order to decrease the risk of infection; for example antibiotics may be prescribed to such patients prior to routine dental work.

Another surgical procedure for the treatment of hydrocephalus is third ventriculostomy. In this procedure, the floor of the third ventricle is punctured or fenestrated to allow excess CSF to flow from the ventricles to the sub-arachnoid space, whence the CSF may undergo reabsorption. In third ventriculostomy of the prior art, the procedure typically entails passing an endoscope through a burr hole into the third ventricle, and forming a hole in the floor of the third ventricle. In conventional ventriculostomy procedures the hole is typically formed mechanically, for example using a guide wire, closed forceps, or the endoscope itself. The hole may then be enlarged using a balloon catheter.

Third ventriculostomy of the prior art suffers from a number of drawbacks and disadvantages. For example, the hole formed in the third ventricle, or stents inserted therethrough, may become occluded. (See, for example, G. Cinalli, et al., Failure of Third Ventriculostomy in the Treatment of Aqueductal Stenosis in Children, Journal of Neurosurgery, Vol. 90, 1999.) In addition, the process of penetrating the floor of the third ventricle using mechanical devices can cause damage to the ventricles, and may cause excessive bleeding. Furthermore, attempts to prevent bleeding using monopolar coagulation instruments can increase the temperature of the CSF within the third ventricle to excessively high levels.

Lasers have been used in a number of surgical applications to ablate or vaporize target tissue. Unfortunately, lasers are both expensive and somewhat tedious to use. Another disadvantage with lasers is the difficulty in judging the depth of tissue ablation. In order to avoid inadvertent damage or destruction to underlying or surrounding non-target tissue, it is often essential to control the depth of ablation. This is particularly important when performing procedures in highly sensitive areas in or around the central nervous system. However, because the surgeon generally points and shoots a laser without contacting the tissue, he or she does not receive any tactile feedback to judge how deeply the laser is cutting.

Monopolar radiofrequency (RF) devices have been used for conventional electrosurgical removal, cutting, and/or coagulation of tissue. Monopolar devices suffer from the disadvantage that the electric current will flow through undefined paths in the patient's body, thereby increasing the risk of undesirable electrical stimulation to portions of the patient's body. In addition, since the defined path through the patient's body has a relatively high impedance (because of the large distance or resistivity of the patient's body), large voltage differences must typically be applied between the return and active electrodes in order to generate a current suitable for ablation or cutting of the target tissue. This current, however, may inadvertently flow along paths within the patient's body having less impedance than the defined electrical path, which will substantially increase the current flowing through these paths, possibly causing damage to or destroying surrounding non-target tissue or neighboring peripheral nerves.

Other disadvantages of conventional RF devices, particularly monopolar devices, include nerve stimulation and interference with monitoring equipment in the operating room. In addition, these devices typically operate by creating a voltage difference between the active electrode and the target tissue, causing an electrical arc to form across the physical gap between the electrode and tissue. At the point of contact of the electric arcs with tissue, rapid tissue heating occurs due to high current density between the electrode and tissue. This high current density causes cellular fluids to rapidly vaporize into steam, thereby producing a “cutting effect” along the pathway of localized tissue heating. Thus, the tissue is parted along the pathway of evaporated cellular fluid, inducing undesirable collateral tissue damage in regions surrounding the target tissue site. This collateral tissue damage often causes indiscriminate destruction of tissue, resulting in the loss of the proper function of the tissue. In addition, the device does not remove any tissue directly, but rather depends on destroying a zone of tissue and allowing the body to eventually remove the destroyed tissue.

Thus, there is a need for bipolar electrosurgical apparatus and methods for fenestrating the third ventricle for the treatment of hydrocephalus, wherein a stoma or hole is formed in the third ventricle by volumetric removal of tissue at a relatively low temperature, and wherein the target site undergoes simultaneous hemostasis.

### SUMMARY OF THE INVENTION

The present invention provides systems, apparatus, and methods for selectively applying electrical energy to structures within a patient's body. The systems and methods of the present invention are useful for shrinkage, ablation, resection, aspiration, and/or hemostasis of tissue and other body structures in open and endoscopic spine surgery. In particular, the present invention includes apparatus and methods for electrosurgical third ventriculostomy for the treatment of hydrocephalus.

Systems according to the present invention generally include an electrosurgical instrument (such as a probe or catheter) having a shaft with proximal and distal ends, an electrode assembly at the distal end, and one or more connectors for coupling the electrode assembly to a source of high frequency electrical energy (i.e., a power supply or generator). The probe or catheter may assume a wide variety of configurations, with the primary purpose being to introduce the electrode assembly to the target site or tissue, and to permit the surgeon to manipulate the electrode assembly from a proximal end of the shaft. The electrode assembly includes one or more active electrode(s) configured for tissue ablation, and/or coagulation, and a return electrode spaced from the active electrode(s). The return electrode may be either on the instrument shaft or separate from the instrument shaft.

In one aspect of the invention, a method is disclosed for fenestrating a wall or floor of the third ventricle (third ventriculostomy) using an electrosurgical instrument. Third ventriculostomy may be used in the treatment of both obstructive hydrocephalus and communicating hydrocephalus. Typically, the instrument (electrosurgical catheter or electrosurgical probe) has an electrode assembly located at a working (i.e., distal) end of the catheter/probe. The electrode assembly includes at least one active electrode and at least one return electrode. In some embodiments, the electrode assembly may further include a coagulation electrode adapted for coagulating tissue. The instrument is introduced into the third ventricle, and the shaft distal end is guided or steered to a target site. While the active electrode is positioned in at least close proximity to the target site, a high frequency voltage is applied between the active and return electrodes sufficient to fenestrate, or form a stoma in, the boundary (wall or floor) of the third ventricle. The window, stoma, or hole formed in the boundary of the third ventricle allows excess CSF to drain from the ventricles into the sub-arachnoid space. The stoma may be enlarged by lateral translation of the electrode assembly with respect to the stoma. This procedure combines certain advantages of third ventriculostomy (as opposed to shunt procedures), such as shunt independence, with the clinical benefits of ArthroCare's Coblationg technology (ArthroCare Corporation, Sunnyvale, Calif.). Such clinical benefits include reduced thermal injury to surrounding structures and tissue, due to lower temperatures in the vicinity of the instrument working end; simultaneous coagulation/hemostasis to prevent bleeding; increased patency of the stoma; and a simpler, more rapid procedure.

In one embodiment the apparatus includes a flexible or rigid endoscope for the introduction of an electrosurgical catheter to the target site (e.g., the floor of the third ventricle). The electrosurgical catheter may include a flexible shaft that is adapted for being guided or steered to a specific target site within the patient. In another embodiment, the apparatus includes an electrosurgical probe and an introducer device adapted for passing a working end of the probe therethrough. In one embodiment, the probe and introducer device are advanced towards the target site under direct visualization via a strategically located access hole in the patient's cranium. In one embodiment, the access hole in the patient's cranium is formed just anterior to the coronal suture and somewhat off the mid-line to allow direct, or substantially linear, access to the third ventricle via the foramen of Monro. The access hole may be formed using a mechanical burr or electrosurgically.

In another aspect, the present invention provides a method for forming an access hole in the cranium of a patient. In such a method, after preparation of the scalp, an active electrode of an electrosurgical instrument is positioned in at least close proximity to the cranium at the target location. Thereafter, a high frequency voltage is applied between the active electrode and a return electrode from a high frequency power supply or electrosurgical generator, wherein the power supply is operating in an ablation mode. The high frequency voltage is sufficient to ablate or remove the bone tissue at the target location through molecular dissociation or disintegration processes. An access hole formed according to the invention may be located at any desired location of the cranium. For example, for a third ventriculostomy procedure the hole may be formed up to about 5 cm anterior to the coronal suture and from about 1 cm to 5 cm from the mid-line. Such an access hole in the cranium may be the size of a standard burr hole, for example from about 10 mm to 14 mm, or may be somewhat smaller, e.g., in the range of from about 2 mm to 8 mm in diameter. Access holes in this size range are suitable for providing access to the brain during various endoscopic or microendoscopic neurosurgical procedures.

In general, ablation of bone requires a higher voltage as compared with that required for removal of soft tissue. Voltage levels for performing various procedures are presented hereinbelow. Typically, for removal of bone from the cranium, an electrically conductive fluid (e.g., electrically conductive gel, isotonic saline) is located between the active electrode and the tissue. In one aspect, the method may include placement of an electrically conductive gel at the target site, e.g., prior to positioning the active electrode with respect to the target location. The applied voltage causes the formation of an ionized vapor or plasma adjacent to the active electrode, and charged particles (e.g., electrons) cause the molecular dissociation or disintegration of the target tissue (e.g., skin and underlying cranium at the target location). This molecular dissociation is accompanied by the volumetric removal of the tissue. This process can be precisely controlled to effect the volumetric removal of tissue as thin as 10 microns to 150 microns, while minimizing or avoiding heating or damaging underlying non-target tissue (e.g., the dura mater). A more complete description of this phenomenon, known as Coblationg, is described in commonly assigned U.S. Pat. No. 5,697,882, the complete disclosure of which is incorporated herein by reference.

According to one aspect, an electrosurgical system of the invention includes a power supply, coupled to the active and return electrodes, for applying a high frequency voltage therebetween. In one embodiment, the system comprises a voltage reduction element coupled between the power supply and active electrode to control the voltage delivered to the active electrode. The voltage reduction element will typically comprise a passive element, such as a capacitor, resistor, inductor, or the like. In one embodiment, the power supply can apply a voltage of about 150 volts RMS to 600 volts RMS between the active and return electrodes, but the voltage reduction element will typically reduce this voltage to about 20 volts RMS to 300 volts RMS to the active electrode. In this manner, the voltage delivered to the active electrode may be reduced below the threshold for ablation of tissue (sub-ablation mode), but the lower voltage is nevertheless sufficient to heat, shrink, stiffen, or coagulate the tissue.

The active electrode(s) may comprise a single active electrode, or an electrode array, extending from an electrically insulating electrode support member or spacer. The support or spacer typically comprises an inorganic material such as a ceramic, a polyimide, a silicone rubber, or a glass. The active electrode will usually have a smaller exposed surface area than the return electrode, such that the current densities are much higher at the active electrode than at the return electrode. The return electrode may have a relatively large, smooth surface configured to reduce current densities thereat, thereby minimizing damage to adjacent non-target tissue.

The apparatus may further include a fluid delivery element for delivering electrically conductive fluid to the active electrode(s) and the target site. The fluid delivery element may be located on the instrument, e.g., a fluid lumen or tube, or it may be part of a separate instrument. Alternatively, an electrically conductive gel or spray, such as isotonic saline or a conductive gel, may be applied to the electrode assembly or to the target site in various ways. In the latter situation, the apparatus may lack a fluid delivery element. Regardless of the manner of delivery, the electrically conductive fluid is delivered so as to provide a current flow path between the active electrode(s) and the return electrode(s).

In another aspect, the present invention provides a method of establishing patency in an occluded cerebral aqueduct for the treatment of aqueductal stenosis using an electrosurgical system. Typically, the electrosurgical system includes a power supply coupled to at least one active electrode disposed on a shaft distal end of an electrosurgical catheter. The method comprises advancing the distal end of the catheter into the third ventricle, and thereafter guiding the catheter into the cerebral aqueduct, such that the active electrode is positioned in at least close proximity to an occlusive material of the cerebral aqueduct. While the active electrode is so positioned, at least a first high frequency voltage is applied to the active electrode, sufficient to volumetrically remove at least a portion of the occlusive material, whereby a channel is formed and patency of the cerebral aqueduct is established or re-established. In a further step, tissue adjacent to the channel may be coagulated to stiffen the adjacent tissue in order to promote patency of the channel over a more prolonged period of time. As an example, the adjacent tissue may be stiffened by applying a second, lower, high frequency voltage to the active electrode in a sub-ablation mode, such that the tissue undergoes shrinkage and/or stiffening due to controlled thermal heating.

For a further understanding of the nature and advantages of the invention, reference should be made to the following description taken in conjunction with the accompanying drawings.

The following commonly assigned provisional applications, patent applications, and patents are related to Coblation® technology used in nervous system applications and are all incorporated by reference: Issued U.S. Pat. Nos. 6,045,532; 6,264,650; 6,264,651; 6,277,112; 6,283,961; 6,322,549; 6,464,695; 6,468,274; and 6,468,270; Pending U.S. application Ser. Nos. 09/676,194 filed Sep. 28, 2000; 09/679,394 filed Oct. 3, 2000; 09/747,311 filed Dec. 20, 2000; 09/665,441 filed Sep. 19, 2000; 09/765,832 filed Jan. 19, 2001; and 09/848,843 filed May 3, 2001; and Provisional application No. 60/408,967

### BRIEF DESCRIPTION OF THE DRAWINGS

FIG. 1 is a perspective view of an electrosurgical system incorporating a power supply and an electrosurgical probe for tissue ablation, resection, incision, contraction and for vessel hemostasis, according to the present invention;

FIG. 2 schematically illustrates one embodiment of a power supply, according to the present invention;

FIG. 3 illustrates an electrosurgical system incorporating a plurality of active electrodes and associated current limiting elements;

FIG. 4 is a side view of an electrosurgical probe according to the present invention;

FIG. 5 is a view of the distal end portion of the probe of FIG. 4;

FIG. 6 is an exploded view of a proximal portion of the electrosurgical probe;

FIGS. 7A and 7B are perspective and end views, respectively, of an alternative electrosurgical probe incorporating an inner fluid lumen;

FIGS. 8A-8C are cross-sectional views of the distal portions of three different embodiments of an electrosurgical probe, according to the present invention;

FIGS. 9-12 are end views of alternative embodiments of the probe of FIG. 4, incorporating aspiration electrode(s);

FIG. 13 shows a longitudinal section of the shaft distal portion of a probe having an aspiration electrode within an aspiration lumen, according to another embodiment of the present invention;

FIGS. 14A-14C illustrate an alternative embodiment incorporating a screen electrode;

FIGS. 15A-15D illustrate four embodiments of electrosurgical probes specifically designed for treating spinal defects;

FIG. 16 illustrates an electrosurgical system incorporating a dispersive return pad for monopolar and/or bipolar operations;

FIG. 17 illustrates a catheter system for electrosurgical treatment of intervertebral discs according to the present invention;

FIGS. 18-22 illustrate a method of performing a microendoscopic discectomy according to the principles of the present invention;

FIG. 23 schematically represents an electrosurgical instrument suitable for forming a hole in a cranium of a patient, according to the instant invention;

FIG. 24 schematically represents formation of a hole in a cranium of a patient using an electrosurgical instrument, according to one embodiment of the invention;

FIG. 25 is a superior view of the cranium showing the location of the midline and coronal suture in relation to the frontal bone and the parietal bone;

FIG. 26 shows the location of the third ventricle in relation to the interpeduncular cistern of the sub-arachnoid space;

FIG. 27 is a side view of the distal end portion of an electrosurgical catheter, according to one embodiment of the invention;

FIGS. 28A-D each represents a side view of an electrosurgical instrument for localized ablation or fenestration of tissue, according to the invention;

FIGS. 29A and 29B illustrate a method of fenestrating the third ventricle, according to one embodiment of the invention;

FIG. 30 shows an electrosurgical catheter introduced into the cerebral aqueduct for establishing patency therein, according to one embodiment of the invention;

FIG. 31 schematically represents a series of steps involved in a method of forming an access hole in the cranium of a patient, using an electrosurgical probe, according to the present invention;

FIG. 32 schematically represents a series of steps involved in a method of performing a third ventriculostomy, according to one embodiment of the invention; and

FIG. 33 schematically represents a series of steps involved in a method of establishing patency in the cerebral aqueduct of a hydrocephalus patient, according to one embodiment of the invention.

### DESCRIPTION OF SPECIFIC EMBODIMENTS

The present invention provides systems and methods for selectively applying electrical energy to a target location within or on a patient's body, particularly including tissue in or around the central nervous system. These procedures include treating interspinous tissue, degenerative discs, and third ventriculostomies. These procedures may be performed under direct visualization, fluoroscopy, or using endoscopy, or the like.

The present invention includes systems and methods which, in one aspect, may be used for treating interspinous tissue. In another aspect, apparatus and methods of the invention may be used for treating the cerebral ventricles. In yet another aspect of the invention, apparatus and methods are provided for treating the cerebral aqueduct. In some embodiments, RF energy is used to ablate, heat, coagulate, stiffen, or shrink a target tissue in or around the brain for the treatment of hydrocephalus. In one aspect of the invention, an active electrode is positioned adjacent to the target tissue and the target tissue is heated, preferably with RF energy, to a sufficient temperature to ablate, stiffen, or coagulate the tissue. In a specific embodiment, a high frequency voltage difference is applied between one or more active electrode(s) and one or more return electrode(s) to develop high electric field intensities in the vicinity of the target tissue to controllably heat the target tissue.

In one aspect of the invention, the target tissue, e.g., a defined region, or target site, in the floor of the third ventricle is volumetrically removed or ablated to form a stoma, or hole within the floor of the third ventricle at the target site . In this procedure, a high frequency voltage is applied between one or more active electrode(s) and one or more return electrode(s) to develop high electric field intensities in the vicinity of the target tissue. The high electric field intensities adjacent the active electrode(s) lead to electric field induced molecular breakdown of target tissue through molecular dissociation (rather than thermal evaporation or carbonization). Applicant believes that the tissue structure is volumetrically removed through molecular disintegration of larger organic molecules into smaller molecules and/or atoms, such as hydrogen, oxygen, oxides of carbon, hydrocarbons and nitrogen compounds. This molecular disintegration completely removes the tissue structure, as opposed to prior art electrosurgical desiccation and vaporization of tissue which typically involve dehydrating the tissue by the removal of water from within the cells and extracellular fluids.

The high electric field intensities may be generated by applying a high frequency voltage that is sufficient to vaporize an electrically conductive fluid over at least a portion of the active electrode(s) in the region between the distal tip of the active electrode(s) and the target tissue. The electrically conductive fluid may be a gas, a gel, or a liquid, such as isotonic saline, blood, or cerebrospinal fluid. Since the vapor layer or vaporized region has a relatively high electrical impedance, it minimizes current flow into the electrically conductive fluid. This ionization, under the conditions described herein, induces the discharge of energetic electrons and photons from the vapor layer and to the surface of the target tissue. A more detailed description of this phenomenon, termed Coblation® can be found in commonly assigned U.S. Pat. No. 5,697,882, the complete disclosure of which is incorporated herein by reference.

Applicant believes that the principal mechanism of tissue removal in the Coblation® mechanism of the present invention is molecular dissociation of tissue components induced by energetic electrons or ions that have been energized in a plasma adjacent to the active electrode(s). When a liquid is heated enough that atoms vaporize from the surface faster than they recondense, a gas is formed. When the gas is heated sufficiently that the atoms collide with each other and their electrons are removed in the process, an ionized gas or plasma is formed (the so-called “fourth state of matter”). A more complete description of plasmas can be found in Plasma Physics, by R. J. Goldston and P. H. Rutherford of the Plasma Physics Laboratory of Princeton University (1995), the entire contents of which are incorporated herein by reference. When the density of the vapor layer (or within a bubble formed in the electrically conductive liquid) becomes sufficiently low (i.e., less than approximately 10²⁰ atoms/cm³ for aqueous solutions), the electron mean free path increases to enable subsequently injected electrons to cause impact ionization within these regions of low density (i.e., vapor layers or bubbles). Once the ionic particles in the plasma layer have sufficient energy, they accelerate towards the target tissue. Energy evolved by the energetic electrons (e.g., 3.5 eV to 5 eV) can subsequently bombard a molecule and break its bonds, dissociating a molecule into free radicals, which then combine into final gaseous or liquid species.

Plasmas may be formed by heating a gas and ionizing the gas by driving an electric current through it, or by transmitting radio waves into the gas. Generally, these methods of plasma formation give energy to free electrons in the plasma directly, and then electron-atom collisions liberate more electrons, and the process cascades until the desired degree of ionization is achieved. Often, the electrons carry the electrical current or absorb the radio waves and, therefore, are hotter than the ions. Thus, in applicant's invention, the electrons, which are carried away from the tissue towards the return electrode, carry most of the plasma's heat with them, allowing the ions to break apart the tissue molecules in a substantially non-thermal manner.

In some embodiments, the present invention applies high frequency (RF) electrical energy in an electrically conductive fluid to shrink (e.g., to decrease the dimensions, tighten, contract, or reduce the volume), coagulate, or ablate (e.g., resect, cut, or volumetrically remove) a target tissue, organ, or structure, and to seal transected vessels (to effect hemostasis) in the region of the target tissue. The present invention may also be useful for sealing larger arterial vessels, e.g., on the order of about 1 mm in diameter.

In some embodiments, a high frequency power supply is provided having an ablation mode, wherein a first voltage is applied to an active electrode sufficient to effect molecular dissociation or disintegration of the tissue, and a coagulation mode, wherein a second, lower voltage is applied to an active electrode (either the same or a different electrode) sufficient to heat, shrink, and/or achieve hemostasis of severed vessels within the tissue. In other embodiments, an electrosurgical instrument is provided having one or more coagulation electrode(s) configured for sealing a severed vessel, such as an arterial vessel; and one or more active electrodes configured for either contracting the collagen fibers within the tissue, or removing (ablating) the tissue, e.g., by applying sufficient energy to the tissue to effect molecular dissociation. In the latter embodiments, the coagulation electrode(s) may be configured such that a single voltage can be applied to coagulate with the coagulation electrode(s), and to ablate or shrink with the active electrode(s). In other embodiments, the power supply is combined with the coagulation instrument such that the coagulation electrode is used when the power supply is in the coagulation mode (low voltage), and the active electrode(s) are used when the power supply is in the ablation mode (higher voltage).

In one method of the present invention, one or more active electrodes are brought into close proximity to tissue at a target site, and the power supply is activated in the ablation mode such that sufficient voltage is applied between the active electrodes and the return electrode to volumetrically remove the tissue through molecular dissociation, as described below. During this process, vessels within the tissue will be severed. Smaller vessels will be automatically sealed with the system and method of the present invention. Larger vessels, and those with a higher flow rate, such as arterial vessels, may not be automatically sealed in the ablation mode. In these cases, the severed vessels may be sealed by activating a control (e.g., a foot pedal) to reduce the voltage of the power supply into the coagulation mode. In this mode, the active electrodes may be pressed against the severed vessel to provide sealing and/or coagulation of the vessel. Alternatively, a coagulation electrode located on the same or a different instrument may be pressed against the severed vessel. Once the vessel is adequately sealed, the surgeon activates a control (e.g., another foot pedal) to increase the voltage of the power supply back into the ablation mode.

In another aspect, the present invention may be used to shrink or contract collagen connective tissue which support the vertebral column, or tissue within the disc. In these procedures, the RF energy heats the tissue directly by virtue of the electrical current flow therethrough, and/or indirectly through the exposure of the tissue to fluid heated by RF energy, to elevate the tissue temperature from normal body temperatures (e.g., 37° C.) to temperatures in the range of 45° C. to 90° C., preferably in the range from about 60° C. to 70° C. Thermal shrinkage of collagen fibers occurs within a small temperature range, which, for mammalian collagen, is in the range from 60° C. to 70° C. (Deak, G., et al., “The Thermal Shrinkage Process of Collagen Fibres as Revealed by Polarization Optical Analysis of Topooptical Staining Reactions,” Acta Morphological Acad. Sci. of Hungary, Vol. 15(2), pp. 195-208, 1967). Previously reported research has attributed thermal shrinkage of collagen to the cleaving of the internal stabilizing cross-linkages within the collagen matrix (Deak, G., ibid). It has also been reported that when the collagen temperature is increased above 70° C., the collagen matrix begins to relax again and the shrinkage effect is reversed resulting in no net shrinkage (Allain, J. C., et al., “Isometric Tensions Developed During the Hydrothermal Swelling of Rat Skin,” Connective Tissue Research, Vol. 7, pp. 127-133, 1980), the complete disclosure of which is incorporated herein by reference. Consequently, the controlled heating of tissue to a precise depth is critical to the achievement of therapeutic collagen shrinkage. A more detailed description of collagen shrinkage can be found in U.S. Pat. No. 6,159,194, the complete disclosure of which is incorporated by reference.

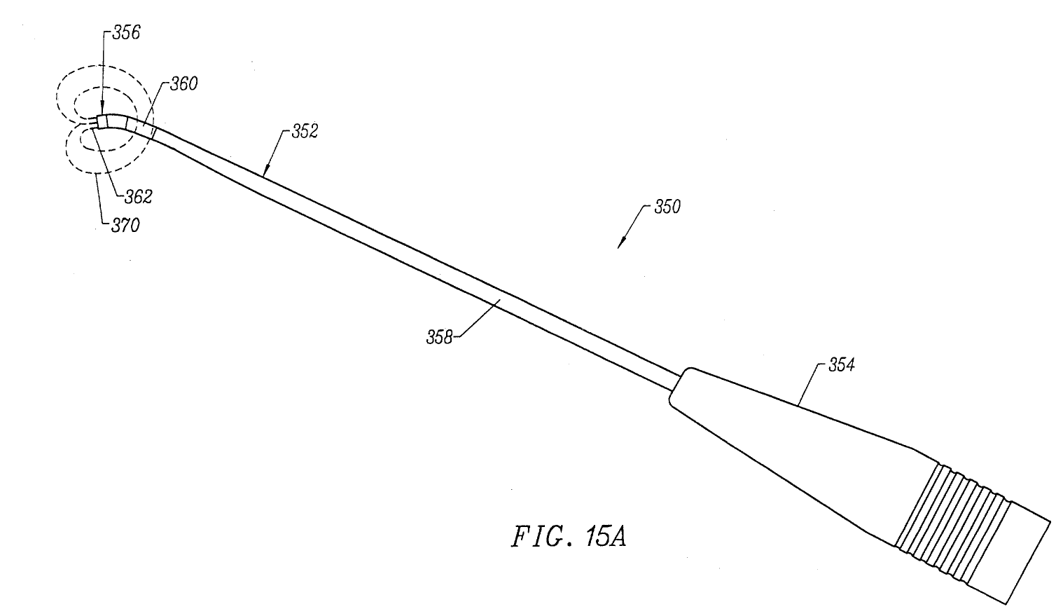

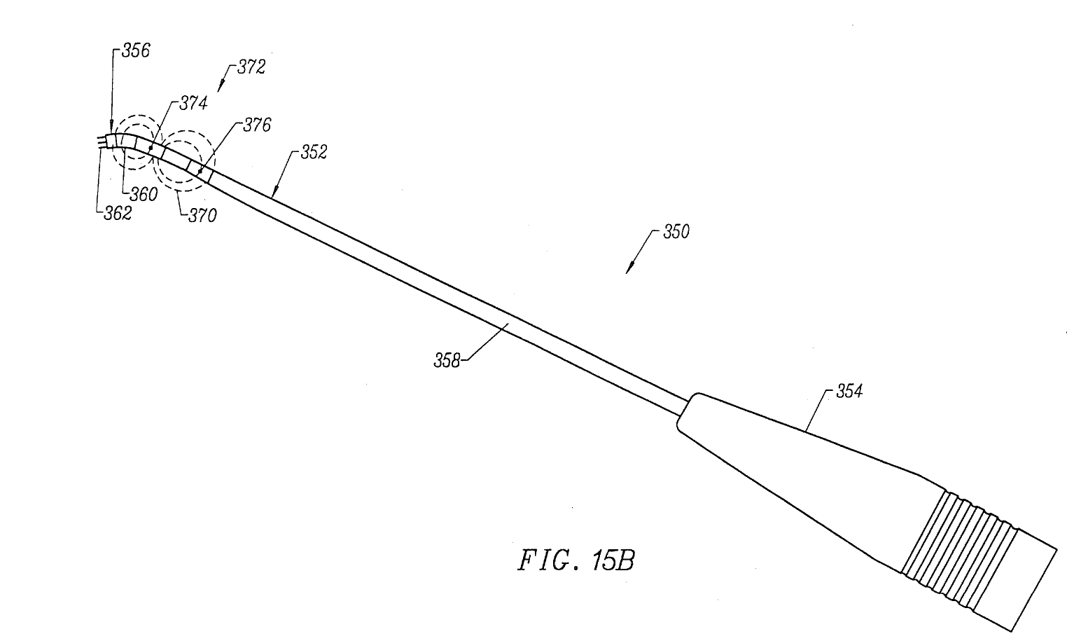

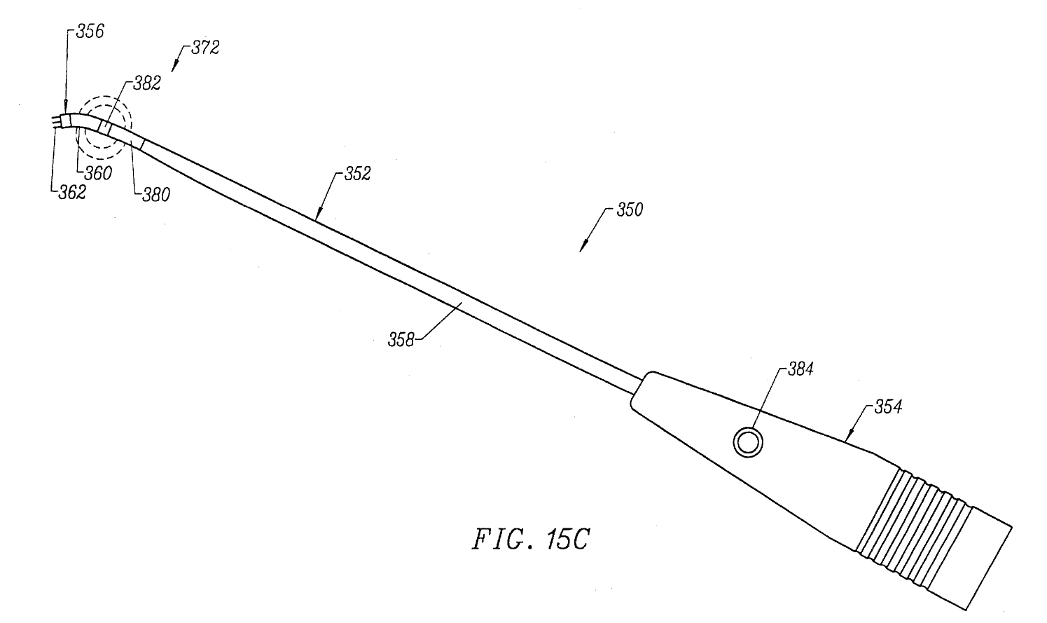

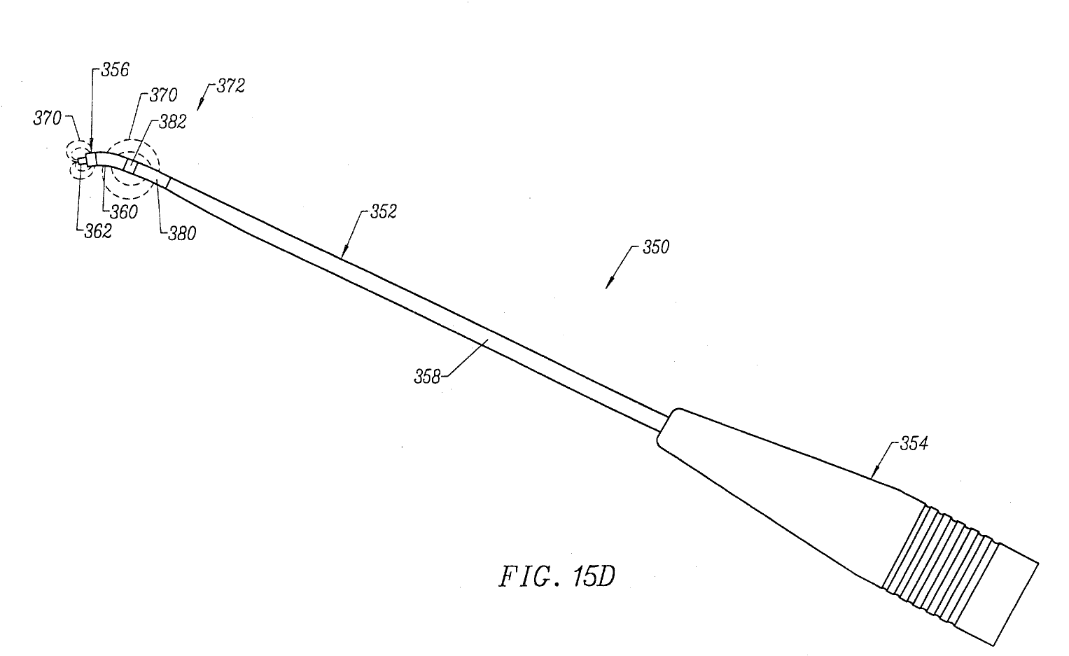

The preferred depth of heating to effect the shrinkage of collagen in the heated region (i.e., the depth to which the tissue is elevated to temperatures between 60° C. to 70° C.) generally depends on (1) the thickness of the target tissue, (2) the location of nearby structures (e.g., nerves) that should not be exposed to damaging temperatures, and/or (3) the location of the collagen tissue layer within which therapeutic shrinkage is to be effected. The depth of heating is usually in the range from 1.0 mm to 5.0 mm. In some embodiments of the present invention, the tissue is purposely damaged in a thermal heating mode to create necrosed or scarred tissue at the tissue surface. The high frequency voltage in the thermal heating mode is below the threshold of ablation as described above, but sufficient to cause some thermal damage to the tissue immediately surrounding the electrodes without vaporizing or otherwise debulking this tissue in situ. Typically, it is desired to achieve a tissue temperature in the range of about 60° C. to 100° C. to a depth of about 0.2 mm to 5 mm, usually about 1 mm to 2 mm. The voltage required for this thermal damage will partly depend on the electrode configurations, the conductivity of the area immediately surrounding the electrodes, the time period in which the voltage is applied and the depth of tissue damage desired. With the electrode configurations described in this application (e.g., FIGS. 15A-15D), the voltage level for thermal heating will usually be in the range of about 20 volts RMS to 300 volts RMS, preferably about 60 volts RMS to 200 volts RMS. The peak-topeak voltages for thermal heating with a square wave form having a crest factor of about 2 are typically in the range of about 40 volts peak-to-peak to 600 volts peak-to-peak, preferably about 120 volts peak-to-peak to 400 volts peak-to-peak. In some embodiments, capacitors or other electrical elements may be used to increase the crest factor up to 10. The higher the voltage is within this range, the less time required. If the voltage is too high, however, the surface tissue may be vaporized, debulked or ablated, which is generally undesirable.

In yet another embodiment, the present invention may be used for electrosurgically forming an access hole in the cranium of a patient about to undergo a neurosurgical procedure. In these embodiments, the active electrode(s) of an electrosurgical probe are positioned in at least close proximity to the cranium at a target location. High frequency voltage is applied between the active electrode(s) and a return electrode to locally ablate the cranium tissue at the target location. In these embodiments, the active electrode(s) are capable of generating high current densities on one or more surfaces thereof, while the return electrode will typically be positioned proximally from the active electrode(s) on the probe shaft. Typically, an electrically conductive fluid is applied to the target site to provide a current flow path between the active and return electrodes. The present invention is also useful for removing or ablating tissue around nerves, such as spinal, peripheral or cranial nerves. One of the significant drawbacks with prior art shavers or microdebriders, conventional electrosurgical devices, and lasers is that they do not differentiate between the target tissue and the surrounding nerves or bone. Therefore, the surgeon must be extremely careful during procedures using these devices to avoid damage to the bone or nerves within and around the target site. In the present invention, the Coblation® process for treating tissue results in no, or minimal, collateral tissue damage, as discussed above. This allows the surgeon to remove tissue close to a nerve without causing collateral damage to the nerve.

In addition to the generally precise nature of the novel mechanisms of the present invention, applicant has discovered an additional method of ensuring that adjacent nerves are not damaged during tissue removal. According to the present invention, systems and methods are provided for distinguishing between the fatty tissue immediately surrounding nerve fibers and the target tissue that is to be removed during the procedure. Peripheral nerves usually comprise a connective tissue sheath, or epineurium, enclosing the bundles of nerve fibers, each bundle being surrounded by its own sheath of connective tissue (the perineurium) to protect these nerve fibers. The outer protective tissue sheath or epineurium typically comprises a fatty tissue (e.g., adipose tissue) having substantially different electrical properties from those of the target tissue, such as cerebral aqueduct or cerebral ventricles. The system of the present invention measures the electrical properties of the tissue at the tip of the probe with one or more active electrode(s). These electrical properties may include electrical conductivity at one, several, or a range of frequencies (e.g., in the range from 1 kHz to 100 MHz), dielectric constant, capacitance or combinations of these. In this embodiment, an audible signal may be produced when the sensing electrode(s) at the tip of the probe detects the fatty tissue surrounding a nerve, or direct feedback control can be provided to only supply power to the active electrode(s) either individually or to the complete array of electrodes, if and when the tissue encountered at the tip or working end of the probe is “normal” (e.g., non-fatty) tissue based on the measured electrical properties.

In one embodiment, the current limiting elements (discussed in detail below) are configured such that the active electrodes will shut down or turn off when the electrical impedance reaches a threshold level. When this threshold level is set to the impedance of the fatty tissue surrounding nerves, the active electrodes will shut off whenever they come in contact with, or in close proximity to, nerves. Meanwhile, the other active electrodes, which are in contact with or in close proximity to target tissue, will continue to conduct electric current to the return electrode. This selective ablation of lower impedance tissue in combination with the Coblation® mechanism of the present invention allows the surgeon to precisely remove tissue around nerves or bone. Applicant has found that the present invention is capable of volumetrically removing tissue closely adjacent to nerves without impairing the function of the nerves, and without significantly damaging the tissue of the epineurium.

In addition to the above, applicant has discovered that the Coblation® mechanism of the present invention can be manipulated to ablate or remove certain tissue structures, while having little effect on other tissue structures. As discussed above, the present invention uses a technique of vaporizing electrically conductive fluid to form a plasma layer or pocket around the active electrode(s), and then inducing the discharge of energy from this plasma or vapor layer to break the molecular bonds of the tissue structure. Energy evolved by the energetic electrons (e.g., 4 eV to 5 eV) can subsequently bombard a molecule and break its bonds, dissociating a molecule into free radicals, which then combine into final gaseous or liquid species. The energy evolved by the energetic electrons may be varied by adjusting a variety of factors, such as: the number of active electrodes; electrode size and spacing; electrode surface area; asperities and sharp edges on the electrode surfaces; electrode materials; applied voltage and power; current limiting means, such as inductors; electrical conductivity of the fluid in contact with the electrodes; density of the fluid; and other factors.

Since different tissue structures have different molecular bonds, the present invention can be configured to break the molecular bonds of certain tissue, while having too low an energy to break the molecular bonds of other tissue. For example, fatty tissue, (e.g., adipose tissue) has double bonds that require a substantially higher energy level than 4 eV to 5 eV to break (typically on the order of about 8 eV). Accordingly, the present invention in its current configuration generally does not ablate or remove such fatty tissue. However, the present invention may be used to effectively ablate cells to release the inner fat content in a liquid form. Of course, factors may be changed such that these double bonds can also be broken in a similar fashion as the single bonds (e.g., increasing voltage or changing the electrode configuration to increase the current density at the electrode tips). A more complete description of this phenomena can be found in commonly assigned U.S. Pat. No. 6,355,032, the complete disclosure of which is incorporated herein by reference.

In yet other embodiments, the present invention provides systems, apparatus and methods for selectively removing tumors, e.g., facial tumors, or other undesirable body structures while minimizing the spread of viable cells from the tumor. Conventional techniques for removing such tumors generally result in the production of smoke in the surgical setting, termed an electrosurgical or laser plume, which can spread intact, viable bacteria or viral particles from the tumor or lesion to the surgical team or to other portions of the patient's body. This potential spread of viable cells or particles has resulted in increased concerns over the proliferation of certain debilitating and fatal diseases, such as hepatitis, herpes, HIV, and papillomavirus. In the present invention, high frequency voltage is applied between the active electrode(s) and one or more return electrode(s) to volumetrically remove at least a portion of the tissue of the tumor or lesion by the dissociation or disintegration of large organic molecules (e.g., proteins and nucleic acids) into non-viable atoms and low molecular species. Specifically, the present invention converts solid tissue into non-condensable gases that are no longer intact or viable, and thus, incapable of spreading viable tumor cells or infectious agents to other portions of the patient's body or to the surgical staff. The high frequency voltage is preferably selected to effect controlled removal of such target tissue while minimizing damage to surrounding or underlying tissue. A more complete description of this phenomenon can be found in co-pending U.S. patent application Ser. No. 09/109,219, filed Jun. 30, 1998, now abandoned, the complete disclosure of which is incorporated herein by reference.

The electrosurgical probe or catheter of the present invention can comprise a shaft or a handpiece having a proximal end and a distal end which supports one or more active electrode(s). The shaft or handpiece may assume a wide variety of configurations, with the primary purpose being to mechanically support the active electrode and permit the treating physician to manipulate the electrode from a proximal end of the shaft. The shaft may be rigid or flexible, with flexible shafts optionally being combined with a generally rigid external tube for mechanical support. Flexible shafts may be combined with pull wires, shape memory actuators, and other known mechanisms for effecting selective deflection of the distal end of the shaft to facilitate positioning of the electrode array. The shaft will usually include a plurality of wires or other conductive elements running axially therethrough to permit connection of the electrode array to a connector at the proximal end of the shaft.

For endoscopic procedures, the shaft will have a suitable diameter and length to allow the surgeon to reach the target site . Thus, the shaft will usually have a length in the range of about 5.0 cm to 30.0 cm, and a diameter in the range of about 0.2 mm to about 20 mm. . The shaft may also be introduced through rigid or flexible endoscopes. Alternatively, the shaft may be a flexible catheter that is introduced through a percutaneous penetration in the patient. Specific shaft designs will be described in detail in connection with the drawings hereinafter.

In one embodiment, the probe may comprise a long, thin needle-like shaft (e.g., on the order of about 1 mm in diameter or less) that can be introduced into the patient percutaneously. The needle will include one or more active electrode(s) for applying electrical energy to the target tissue. The needle may include one or more return electrode(s), or the return electrode may be positioned on the patient as a dispersive pad. In either embodiment, sufficient electrical energy is applied to the active electrode(s) to either shrink the collagen fibers within the tissue, to volumetrically remove at least a portion of the target tissue, to coagulate tissue adjacent to a target site to effect hemostasis, or to stiffen the treated tissue.

The electrosurgical instrument may also be a catheter that is delivered percutaneously and/or endoluminally into the patient by insertion through a conventional or specialized guide catheter, or the invention may include a catheter having an active electrode or electrode array integral with its distal end. The catheter shaft may be rigid or flexible, with flexible shafts optionally being combined with a generally rigid external tube for mechanical support. Flexible shafts may be combined with pull wires, shape memory actuators, and other known mechanisms for effecting selective deflection of the distal end of the shaft to facilitate positioning of the electrode or electrode array. The catheter shaft will usually include a plurality of wires or other conductive elements running axially therethrough to permit connection of the electrode or electrode array and the return electrode to a connector at the proximal end of the catheter shaft. The catheter shaft may include a guide wire for guiding the catheter to the target site, or the catheter may comprise a steerable guide catheter. The catheter may also include a substantially rigid distal end portion to increase the torque control of the distal end portion as the catheter is advanced further into the patient's body. Specific shaft designs will be described in detail in connection with the drawings hereinafter.

The active electrode(s) are preferably supported within or by an insulating support positioned near the distal end of the instrument shaft. The return electrode may be located on the instrument shaft, on another instrument, or on the external surface of the patient (i.e., a dispersive pad). The close proximity of nerves and other sensitive tissue in and around the spinal cord, however, makes a bipolar design more preferable because this minimizes the current flow through non-target tissue and surrounding nerves. Accordingly, the return electrode is preferably either integrated with the instrument body, or with another device located in close proximity to the instrument body. The proximal end of the instrument(s) will include the appropriate electrical connections for coupling the return electrode(s) and the active electrode(s) to a high frequency power supply, such as an electrosurgical generator.

In some embodiments, the active electrode(s) have an active portion or surface with surface geometries shaped to promote high electric field intensity and associated current density along the leading edges of the electrodes. Suitable surface geometries may be obtained by creating electrode shapes that include preferential sharp edges, or by creating asperities or other surface roughness on the surface(s) of the active electrode(s). Electrode shapes according to the present invention can include the use of formed wire (e.g., by drawing round wire through a shaping die) to form electrodes with a variety of cross-sectional shapes, such as square, rectangular, L or V shaped, or the like. Electrode edges may also be created by removing a portion of the elongate metal electrode to reshape the cross-section. For example, material can be ground along the length of a round or hollow wire electrode to form D or C shaped wires, respectively, with edges facing in the cutting direction. Alternatively, material can be removed at closely spaced intervals along the electrode length to form transverse grooves, slots, threads or the like along the electrodes.

Additionally or alternatively, the active electrode surface(s) may be modified through chemical, electrochemical or abrasive methods to create a multiplicity of surface asperities on the electrode surface. These surface asperities will promote high electric field intensities between the active electrode surface(s) and the target tissue to facilitate ablation or cutting of the tissue. For example, surface asperities may be created by etching the active electrodes with etchants having a pH less than 7.0, or by using a high velocity stream of abrasive particles (e.g., grit blasting) to create asperities on the surface of an elongated electrode. A more detailed description of such electrode configurations can be found in U.S. Pat. No. 5,843,019, the complete disclosure of which is incorporated herein by reference.

The return electrode is typically spaced proximally from the active electrode(s) a suitable distance to avoid electrical shorting between the active and return electrodes in the presence of electrically conductive fluid. In some of the embodiments described herein, the distal edge of the exposed surface of the return electrode is spaced about 0.5 mm to 25 mm from the proximal edge of the exposed surface of the active electrode(s), preferably about 1.0 mm to 5.0 mm. Of course, this distance may vary with different voltage ranges, conductive fluids, and depending on the proximity of tissue structures to active and return electrodes. The return electrode will typically have an exposed length in the range of about 1 mm to 20 mm.

The current flow path between the active electrodes and the return electrode(s) may be provided by a body fluid naturally present in situ and surrounding the target site, by submerging the tissue site in an electrical conducting fluid (e.g., within a viscous fluid, such as an electrically conductive gel) or by directing an electrically conductive fluid (e.g., a liquid, such as isotonic saline, hypotonic saline; or a gas, such as argon) to the target site via a fluid delivery element of the instrument. The conductive gel may also be delivered to the target site to achieve a slower, more controlled delivery rate of conductive fluid. In addition, the viscous nature of the gel may allow the surgeon to more easily contain the gel around the target site (e.g., rather than attempting to contain isotonic saline). A more complete description of an exemplary method of directing electrically conductive fluid between the active and return electrodes is described in U.S. Pat. No. 5,697,281, the complete disclosure of which is incorporated herein by reference.

Alternatively, the body's natural conductive fluids, such as blood or cerebrospinal fluid, may be sufficient to establish a conductive path between the return electrode(s) and the active electrode(s), and to provide the conditions for establishing a vapor layer, as described above. In certain applications or procedures, a liquid electrically conductive fluid (e.g., isotonic saline) may be used to concurrently “bathe” the target tissue surface to provide an additional means for removing any tissue, and to cool the region of the target tissue ablated in the previous moment.

The power supply may include a fluid interlock for interrupting power to the active electrode(s) when there is insufficient conductive fluid around the active electrode(s). This ensures that the instrument will not be activated when conductive fluid is not present, minimizing the tissue damage that may otherwise occur. A more complete description of such a fluid interlock can be found in commonly assigned, U.S. Pat. No. 6,235,020, incorporated by reference above.

In some procedures, it may also be necessary to retrieve or aspirate the electrically conductive fluid and/or the non-condensable gaseous products of ablation. In addition, it may be desirable to aspirate small pieces of tissue or other body structures that are not completely disintegrated by the high frequency energy, or other fluids at the target site, such as blood, mucus, etc. Accordingly, the system of the present invention may include one or more suction lumen(s) in the instrument, or on another instrument, coupled to a suitable vacuum source for aspirating fluids from the target site. In addition, the invention may include one or more aspiration electrode(s) coupled to the distal end of the suction lumen for ablating, or at least reducing the volume of, non-ablated tissue fragments that are aspirated into the lumen. The aspiration electrode(s) function mainly to inhibit clogging of the lumen that may otherwise occur as larger tissue fragments are drawn therein. The aspiration electrode(s) may be different from the ablation active electrode(s), or the same electrode(s) may serve both functions. A more complete description of instruments incorporating aspiration electrode(s) can be found in commonly assigned, U.S. Pat. No. 6,190,381, the complete disclosure of which is incorporated herein by reference.

As an alternative or in addition to suction, it may be desirable to contain the excess electrically conductive fluid, tissue fragments, and/or gaseous products of ablation at or near the target site with a containment apparatus, such as a basket, retractable sheath, or the like. This embodiment has the advantage of ensuring that the conductive fluid, tissue fragments, or ablation products do not flow through the patient's vasculature or into other portions of the body. In addition, it may be desirable to limit the amount of suction to limit the undesirable effect suction may have on hemostasis of severed blood vessels.

The present invention may use a single active electrode or an array of active electrodes spaced around the distal surface of a catheter or probe. In the latter embodiment, the electrode array usually includes a plurality of independently current-limited and/or power-controlled active electrodes to apply electrical energy selectively to the target tissue while limiting the unwanted application of electrical energy to the surrounding tissue and environment resulting from power dissipation into surrounding electrically conductive fluids, such as cerebrospinal fluid, normal saline, and the like. The active electrodes may be independently current-limited by isolating the terminals from each other and connecting each terminal to a separate power source that is isolated from the other active electrodes. Alternatively, the active electrodes may be connected to each other at either the proximal or distal ends of the catheter to form a single wire that couples to a power source.

In one configuration, each individual active electrode in the electrode array is electrically insulated from all other active electrodes in the array within the instrument and is connected to a power source which is isolated from each of the other active electrodes in the array or to circuitry which limits or interrupts current flow to the active electrode when low resistivity material (e.g., blood, electrically conductive saline irrigant or electrically conductive gel) causes a lower impedance path between the return electrode and the individual active electrode. The isolated power sources for each individual active electrode may be separate power supply circuits having internal impedance characteristics which limit power to the associated active electrode when a low impedance return path is encountered. By way of example, the isolated power source may be a user selectable constant current source. In this embodiment, lower impedance paths will automatically result in lower resistive heating levels since the heating is proportional to the square of the operating current times the impedance. Alternatively, a single power source may be connected to each of the active electrodes through independently actuatable switches, or by independent current limiting elements, such as inductors, capacitors, resistors and/or combinations thereof. The current limiting elements may be provided in the instrument, connectors, cable, controller or along the conductive path from the controller to the distal tip of the instrument. Alternatively, the resistance and/or capacitance may occur on the surface of the active electrode(s) due to oxide layers which form on certain metals (e.g., titanium), or a resistive coating on the surface of a metal (such as platinum).

The tip region of the instrument may comprise many independent active electrodes designed to deliver electrical energy in the vicinity of the tip. The selective application of electrical energy to the conductive fluid is achieved by connecting each individual active electrode and the return electrode to a power source having independently controlled or current limited channels. The return electrode(s) may comprise a single tubular member of conductive material proximal to the electrode array at the tip. The single tubular member may also serve as a conduit for the supply of an electrically conductive fluid to the active and return electrodes. Alternatively, the instrument may comprise an array of return electrodes at the distal tip of the instrument (together with the active electrodes) to maintain the electric current at the tip. The application of high frequency voltage between the return electrode(s) and the electrode array results in the generation of high electric field intensities at the distal tips of the active electrodes with conduction of high frequency current from each individual active electrode to the return electrode. The current flow from each individual active electrode to the return electrode(s) is controlled by either active or passive means, or a combination thereof, to deliver electrical energy to the surrounding conductive fluid while minimizing energy delivery to surrounding (non-target) tissue.

The application of a high frequency voltage between the return electrode(s) and the active electrode(s) for appropriate time intervals effects shrinking, cutting, removing, ablating, shaping, stiffening, coagulating, contracting, or otherwise modifying the target tissue. In some embodiments of the present invention, the tissue volume over which energy is dissipated (i.e., over which a high current density exists) may be more precisely controlled by, for example, the use of a multiplicity of small active electrodes whose effective diameters or principal dimensions range from about 10 mm to 0.01 mm, preferably from about 2 mm to 0.05 mm, and more preferably from about 1 mm to 0.1 mm. In this embodiment, electrode areas for both circular and non-circular terminals will have a contact area (per active electrode) below 50 mm² for electrode arrays, and as large as 75 mm² for single electrode embodiments. In multiple electrode array embodiments, the contact area of each active electrode is typically in the range from 0.0001 mm² to 1 mm², and more preferably from 0.001 mm² to 0.5 mm². The circumscribed area of the electrode array or active electrode is in the range from 0.25 mm² to 75 mm², preferably from 0.5 mm² to 40 mm². In multiple electrode embodiments, the array will usually include at least two isolated active electrodes, often at least five active electrodes, often greater than 10 active electrodes and even 50 or more active electrodes, disposed over the distal contact surfaces on the shaft. The use of small diameter active electrodes increases the electric field intensity and reduces the extent or depth of tissue heating as a consequence of the divergence of current flux lines which emanate from the exposed surface of each active electrode.

The area of the tissue treatment surface can vary widely, and the tissue treatment surface can assume a variety of geometries, with particular areas and geometries being selected for specific applications. The geometries can be planar, concave, convex, hemispherical, conical, a linear “in-line” array, or virtually any other regular or irregular shape. Most commonly, the active electrode(s) or active electrode array will be formed at the distal tip of the electrosurgical instrument shaft, frequently being planar, disk-shaped, or hemispherical surfaces for use in reshaping procedures, or being linear arrays for use in cutting. Alternatively or additionally, the active electrode(s) may be formed on lateral surfaces of the electrosurgical instrument shaft (e.g., in the manner of a spatula), facilitating access to certain body structures in endoscopic procedures.

It should be clearly understood that the invention is not limited to electrically isolated active electrodes, or even to a plurality of active electrodes. For example, the array of active electrodes may be connected to a single lead that extends through the catheter shaft to a power source of high frequency current. Alternatively, the instrument may incorporate a single electrode that extends directly through the catheter shaft or is connected to a single lead that extends to the power source. The active electrode(s) may have ball shapes (e.g., for tissue vaporization and desiccation), twizzle shapes (for vaporization and needle-like cutting), spring shapes (for rapid tissue debulking and desiccation), twisted metal shapes, annular or solid tube shapes or the like. Alternatively, the electrode(s) may comprise a plurality of filaments, rigid or flexible brush electrode(s) (for debulking a tumor, such as a fibroid, bladder tumor or a prostate adenoma), side-effect brush electrode(s) on a lateral surface of the shaft, coiled electrode(s), or the like.

In some embodiments, the electrode support and the fluid outlet may be recessed from an outer surface of the instrument or handpiece to confine the electrically conductive fluid to the region immediately surrounding the electrode support. In addition, the shaft may be shaped so as to form a cavity around the electrode support and the fluid outlet. This helps to assure that the electrically conductive fluid will remain in contact with the active electrode(s) and the return electrode(s) to maintain the conductive path therebetween. In addition, this will help to maintain a vapor layer and subsequent plasma layer between the active electrode(s) and the tissue at the treatment site throughout the procedure, which reduces the thermal damage that might otherwise occur if the vapor layer were extinguished due to a lack of conductive fluid. Provision of the electrically conductive fluid around the target site also helps to maintain the tissue temperature at desired levels.

In other embodiments, the active electrodes are spaced from the target tissue a sufficient distance to minimize or avoid contact between the tissue and the vapor layer formed around the active electrodes. The ions within the plasma, however, will have sufficient energy, under certain conditions such as higher voltage levels, to accelerate beyond the vapor layer to the tissue. Thus, the bonds of tissue components are dissociated or broken as in previous embodiments, while minimizing the electron flow, and thus the thermal energy, in contact with the tissue.

The electrically conductive fluid should have a minimum threshold conductivity to provide a suitable conductive path between the return electrode and the active electrode(s). The electrical conductivity of the fluid (in units of milliSiemens per centimeter or mS/cm) will usually be greater than 0.2 mS/cm, preferably greater than 2 mS/cm, and more preferably greater than 10 mS/cm. In an exemplary embodiment, the electrically conductive fluid is isotonic saline, which has an electrical conductivity of about 17 mS/cm. Applicant has found that a more conductive fluid, or one with a higher ionic concentration, will usually provide a more aggressive ablation rate. For example, a saline solution with higher levels of sodium chloride than isotonic saline (which is on the order of about 0.9% sodium chloride), e.g., on the order of greater than 1% or between about 3% and 20%, may be desirable. Alternatively, the invention may be used with different types of conductive fluids that increase the power of the plasma layer by, for example, increasing the quantity of ions in the plasma, or by providing ions that have higher energy levels than sodium ions. For example, the present invention may be used with elements other than sodium, such as potassium, magnesium, calcium and other metals in Groups located towards the left side of the Periodic Table. In addition, other electronegative elements may be used in place of chlorine, such as fluorine.

The voltage difference applied between the return electrode(s) and the active electrode(s) will be at high or radio frequency, typically between about 5 kHz and 20 MHz, usually being between about 30 kHz and 2.5 MHz, preferably being between about 50 kHz and 500 kHz, often less than 350 kHz, and often between about 100 kHz and 200 kHz. In some applications, applicant has found that a frequency of about 100 kHz is useful because the tissue impedance is much greater at this frequency. In other applications, such as procedures in or around the heart or head and neck, higher frequencies may be desirable (e.g., 400-600 kHz) to minimize low frequency current flow into the heart or the nerves of the head and neck. The RMS (root mean square) voltage applied will usually be in the range from about 5 volts to 1000 volts, preferably being in the range from about 10 volts to 500 volts, often between about 150 volts to 400 volts depending on the active electrode size, the operating frequency and the operation mode of the particular procedure or desired effect on the tissue (i.e., contraction, coagulation, cutting, or ablation). Typically, the peak-to-peak voltage for ablation or cutting, with a square wave form, will be in the range of 10 volts to 2000 volts, and preferably in the range of 100 volts to 1800 volts, and more preferably in the range of about 300 volts to 1500 volts, often in the range of about 300 volts to 800 volts peak to peak (again, depending on the electrode size, number of electrodes, the operating frequency, and the operation mode). Lower peak-to-peak voltages will be used for tissue coagulation, thermal heating of tissue, or collagen contraction, and will typically be in the range from 50 to 1500, preferably 100 to 1000 and more preferably 120 to 400 volts peak-topeak (again, these values are computed using a square wave form). Higher peak-to-peak voltages, e.g., greater than about 800 volts peak-to-peak, may be desirable for ablation of harder material, such as bone, depending on other factors, such as the electrode geometries and the composition of the conductive fluid.

As discussed above, the voltage is usually delivered in a series of voltage pulses or alternating current of time varying voltage amplitude, with a sufficiently high frequency (e.g., on the order of 5 kHz to 20 MHz) such that the voltage is effectively applied continuously (as compared with e.g., lasers claiming small depths of necrosis, which are generally pulsed about 10 Hz to 20 Hz). In addition, the duty cycle (i.e., cumulative time in any one-second interval that energy is applied) is on the order of about 50% for the present invention, as compared with pulsed lasers which typically have a duty cycle of about 0.0001%.

The preferred power source of the present invention delivers a high frequency current selectable to generate average power levels ranging from several milliwatts to tens of watts per electrode, depending on the volume of target tissue being treated, and/or the maximum allowed temperature selected for the instrument tip. The power source allows the user to select the voltage level according to the specific requirements of a particular neurosurgery procedure, cardiac surgery, arthroscopic surgery, dermatological procedure, ophthalmic procedures, open surgery, or other endoscopic surgery procedure. For cardiac procedures, and potentially for neurosurgery, the power source may have an additional filter, for filtering leakage voltages at frequencies below 100 kHz, particularly voltages around 60 kHz. Alternatively, a power source having a higher operating frequency, e.g., 300 kHz to 600 kHz may be used in certain procedures in which stray low frequency currents may be problematic. A description of one suitable power source can be found in commonly assigned U.S. Pat. Nos. 6,142,992 and 6,235,020, the complete disclosure of both applications are incorporated herein by reference for all purposes.

The power source may be current limited or otherwise controlled so that undesired heating of the target tissue or surrounding (non-target) tissue does not occur. In a presently preferred embodiment of the present invention, current limiting inductors are placed in series with each independent active electrode, where the inductance of the inductor is in the range of 10 uH to 50,000 uH, depending on the electrical properties of the target tissue, the desired tissue heating rate and the operating frequency. Alternatively, capacitor-inductor (LC) circuit structures may be employed, as described previously in U.S. Pat. No. 5,697,909, the complete disclosure of which is incorporated herein by reference. Additionally, current limiting resistors may be selected. Preferably, these resistors will have a large positive temperature coefficient of resistance so that, as the current level begins to rise for any individual active electrode in contact with a low resistance medium (e.g., saline irrigant or blood), the resistance of the current limiting resistor increases significantly, thereby minimizing the power delivery from the active electrode into the low resistance medium (e.g., saline irrigant or blood).

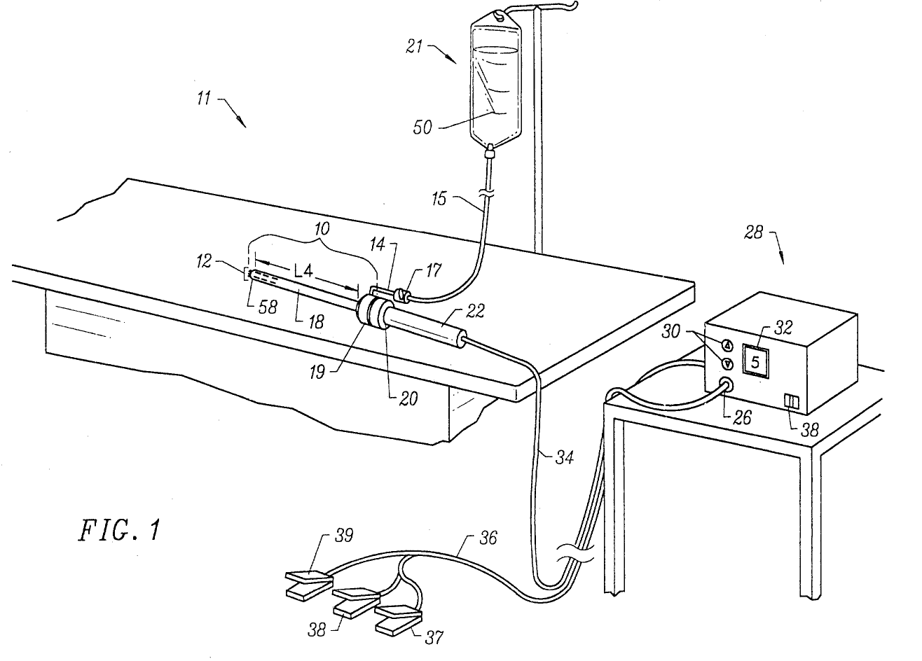

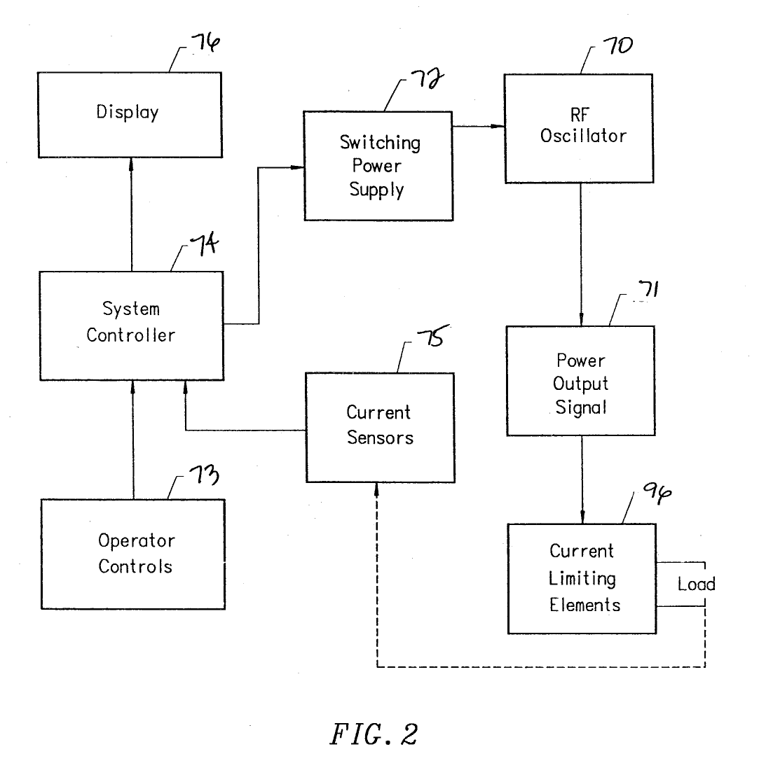

Referring to FIG. 1, an exemplary electrosurgical system 11 according to one embodiment of the invention will now be described in detail. Electrosurgical system 11 generally comprises an electrosurgical handpiece or probe 10 connected to a power supply 28 for providing high frequency voltage to a target site, and a fluid source 21 for supplying electrically conductive fluid 50 to probe 10. In addition, electrosurgical system 11 may include an endoscope (not shown) with a fiber optic head light for viewing the surgical site. The endoscope may be integral with probe 10, or it may be part of a separate instrument. The system 11 may also include a vacuum source (not shown) for coupling to a suction lumen or tube 205 (see FIG. 2) in probe 10 for aspirating the target site.

As shown, probe 10 generally includes a proximal handle 19 and an elongate shaft 18 having an array 12 of active electrodes 58 at its distal end. A connecting cable 34 has a connector 26 for electrically coupling active electrodes 58 to power supply 28. The active electrodes 58 are electrically isolated from each other and each of terminal of active electrodes 58 is connected to an active or passive control network within power supply 28 by means of a plurality of individually insulated conductors (not shown). A fluid supply tube 15 is connected to a fluid tube 14 of probe 10 for supplying electrically conductive fluid 50 to the target site. Fluid supply tube 15 may be connected to a suitable pump (not shown), if desired.

Power supply 28 has an operator controllable voltage level adjustment 30 to change the applied voltage level, which is observable at a voltage level display 32. Power supply 28 also includes first, second and third foot pedals 37, 38, 39 and a cable 36 which is removably coupled to power supply 28. The foot pedals 37, 38, 39 allow the surgeon to remotely adjust the energy level applied to active electrodes 58. In an exemplary embodiment, first foot pedal 37 is used to place the power supply into the “ablation” mode and second foot pedal 38 places power supply 28 into the “sub-ablation” mode (e.g., for coagulation or contraction of tissue). The third foot pedal 39 allows the user to adjust the voltage level within the “ablation” mode. In the ablation mode, a sufficient voltage is applied to the active electrodes to establish the requisite conditions for molecular dissociation of the tissue (i.e., vaporizing a portion of the electrically conductive fluid, forming charged particles within the vapor layer, and accelerating these charged particles against the tissue). As discussed above, the requisite voltage level for ablation will vary depending on the number, size, shape and spacing of the electrodes, the distance to which the electrodes extend from the support member, etc. Once the surgeon places the power supply in the “ablation” mode, voltage level adjustment 30 or third foot pedal 39 may be used to adjust the voltage level to adjust the degree or aggressiveness of the ablation.

Of course, it will be recognized that the voltage and modality of the power supply may be controlled by other input devices. However, applicant has found that foot pedals are convenient methods of controlling the power supply while manipulating the probe during a surgical procedure.

In the sub-ablation mode, the power supply 28 applies a low enough voltage to the active electrodes to avoid vaporization of the electrically conductive fluid and subsequent molecular dissociation of the tissue. The surgeon may automatically toggle the power supply between the ablation and sub-ablation modes by alternately stepping on foot pedals 37, 38, respectively. In some embodiments, this allows the surgeon to quickly move between coagulation/thermal heating and ablation in situ, without having to remove his/her concentration from the surgical field or without having to request an assistant to switch the power supply. By way of example, as the surgeon is treating a target tissue in the ablation mode, the probe typically will simultaneously seal and/or coagulate small severed vessels within the tissue. However, larger vessels, or vessels with high fluid pressures (e.g., arterial vessels) may not be sealed in the ablation mode. Accordingly, the surgeon can simply step on foot pedal 38, automatically lowering the voltage level below the threshold level for ablation, and apply sufficient pressure onto the severed vessel for a sufficient period of time to seal and/or coagulate the vessel. After this is completed, the surgeon may quickly move back into the ablation mode by stepping on foot pedal 37.

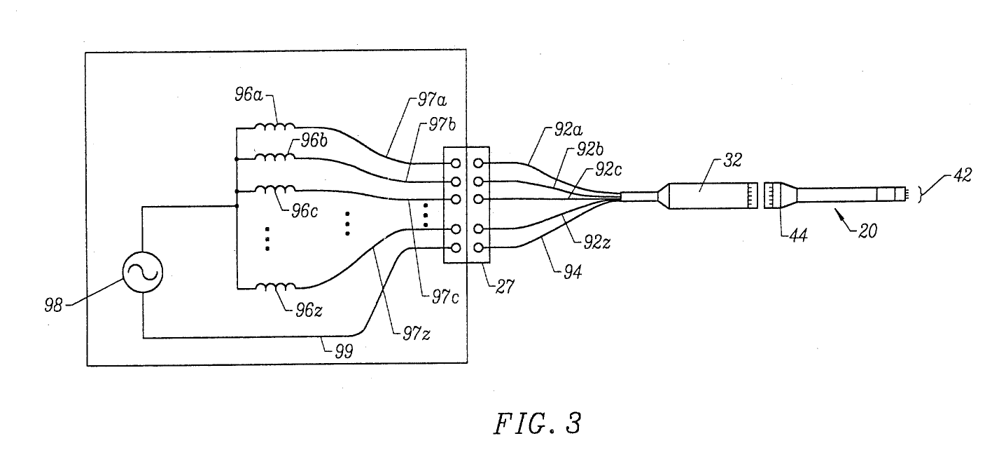

Referring now to FIGS. 2 and 3, a representative high frequency power supply or generator for use according to the principles of the present invention will now be described. The high frequency power supply of the present invention is configured to apply a high frequency voltage of about 10 volts RMS to 500 volts RMS between one or more active electrodes (and/or a coagulation electrode) and one or more return electrodes. In the exemplary embodiment, the power supply applies about 70 volts RMS to 350 volts RMS in the ablation mode, and about 20 volts to 90 volts in a sub-ablation mode, preferably 45 volts to 70 volts in the sub-ablation mode (these values will, of course, vary depending on the probe configuration attached to the power supply and the desired mode of operation).

The preferred power source of the present invention delivers a high frequency current selectable to generate average power levels ranging from several milliwatts to tens of watts per electrode, depending on the volume of target tissue being treated, and/or the maximum allowed temperature selected for the probe tip. The power source allows the user to select the voltage level according to the specific requirements of a particular procedure, e.g., spinal surgery, neurosurgery, arthroscopic surgery, dermatological procedure, ophthalmic procedures, open surgery, or other endoscopic surgery procedure.

As shown in FIG. 2, the power supply or generator generally comprises a radio frequency (RF) power oscillator 70 having output connections for coupling via a power output signal 71 to the load impedance, which is represented by the electrode assembly when the electrosurgical probe is in use. In the representative embodiment, RF oscillator 70 operates at about 100 kHz. The RF oscillator 70 is not limited to this frequency and may operate at frequencies of about 300 kHz to 600 kHz. In particular, for cardiac applications, the RF oscillator will preferably operate in the range of about 400 kHz to about 600 kHz. The RF oscillator will generally supply a square wave signal with a crest factor of about 1 to 2. Of course, this signal may be a sine wave signal or other suitable wave signal depending on the application and other factors, such as the voltage applied, the number and geometry of the electrodes, etc. The power output signal 71 is designed to incur minimal voltage decrease (i.e., sag) under load. This improves the applied voltage to the active electrodes and the return electrode, which improves the rate of volumetric removal of tissue during a procedure involving ablation.

Power is supplied to the oscillator 70 by a switching power supply 72 coupled between the power line and the RF oscillator rather than a conventional transformer. Switching power supply 72 allows the generator to achieve high peak power output without the large size and weight of a bulky transformer. The architecture of switching power supply 72 has also been designed to reduce electromagnetic noise such that U.S. and foreign EMI requirements are met. This architecture comprises a zero voltage switching or crossing, which causes the transistors to turn ON and OFF when the voltage is zero. Therefore, the electromagnetic noise produced by the transistors switching is vastly reduced. In an exemplary embodiment, the switching power supply 72 operates at about 100 kHz.

A system controller 74 coupled to the operator controls 73 (e.g., foot pedals and voltage selector) and display 76, is connected to a control input of switching power supply 72 for adjusting the generator output power by supply voltage variation. The controller 74 may be a microprocessor or an integrated circuit. The generator may also include one or more current sensors 75 for detecting the output current. The power supply is preferably housed within a metal casing which provides a durable enclosure for the electrical components therein. In addition, the metal casing reduces the electromagnetic noise generated within the power supply because the grounded metal casing functions as a “Faraday shield,” thereby shielding the environment from internal sources of electromagnetic noise.

The power supply generally comprises a main or mother board containing generic electrical components required for many different surgical procedures (e.g., arthroscopy, urology, general surgery, dermatology, neurosurgery, etc.), and a daughter board containing application specific current-limiting circuitry (e.g., inductors, resistors, capacitors, and the like). The daughter board is coupled to the mother board by a detachable multi-pin connector to allow convenient conversion of the power supply to, e.g., applications requiring a different current limiting circuit design. For arthroscopy, for example, the daughter board preferably comprises a plurality of inductors of about 200 to 400 microhenries, usually about 300 microhenries, for each of the channels supplying current to the active electrodes (see FIG. 2).

Alternatively, in one embodiment, current limiting inductors are placed in series with each independent active electrode, where the inductance of the inductor is in the range of 10 uH to 50,000 uH, depending on the electrical properties of the target tissue, the desired tissue heating rate, and the operating frequency. Alternatively, capacitor-inductor (LC) circuit structures may be employed, as described previously in co-pending PCT application No. PCT/US94/05168, the complete disclosure of which is incorporated herein by reference. Additionally, current limiting resistors may be selected. Preferably, these resistors will have a large positive temperature coefficient of resistance so that, as the current level begins to rise for any individual active electrode in contact with a low resistance medium (e.g., saline irrigant or conductive gel), the resistance of the current limiting resistor increases significantly, thereby minimizing the power delivery from the active electrode into the low resistance medium (e.g., saline irrigant or conductive gel). Power output signal may also be coupled to a plurality of current limiting elements 96 (FIG. 3), which are preferably located on the daughter board since the current limiting elements may vary depending on the application. A more complete description of a representative power supply can be found in commonly assigned U.S. Pat. No. 6,142,992 incorporated by reference above.

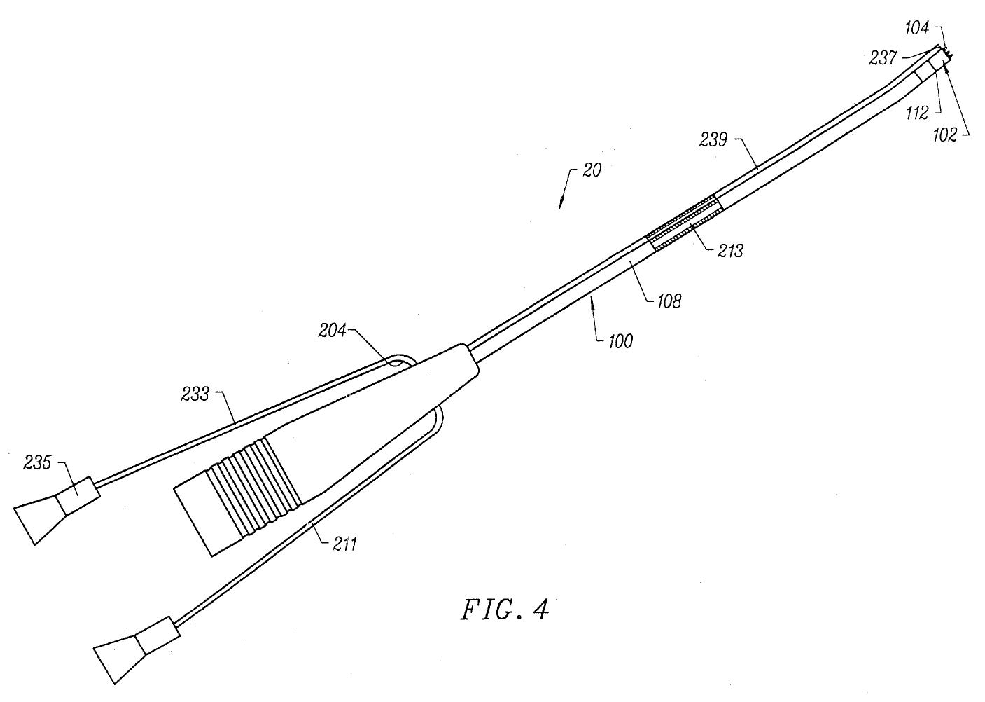

FIGS. 4-6 illustrate an exemplary electrosurgical probe 20 constructed according to the principles of the present invention. As shown in FIG. 4, probe 20 generally includes an elongated shaft 100 which may be flexible or rigid, a handle 204 coupled to the proximal end of shaft 100 and an electrode support member 102 coupled to the distal end of shaft 100. Shaft 100 preferably comprises an electrically conducting material, usually metal, such as tungsten, stainless steel alloys, platinum or its alloys, titanium or its alloys, molybdenum or its alloys, and nickel or its alloys. In this embodiment, shaft 100 includes an electrically insulating jacket 108, which is typically formed as one or more electrically insulating sheaths or coatings, such as polytetrafluoroethylene, polyimide, and the like. The provision of the electrically insulating jacket over the shaft prevents direct electrical contact between these metal elements and any adjacent body structure or the surgeon. Such direct electrical contact between a body structure and an exposed electrode could result in unwanted heating and necrosis of the non-target structure at the point of contact. A return electrode 112 may comprise an annular band coupled to an insulating shaft and having a connector extending within the shaft to the shaft proximal end.

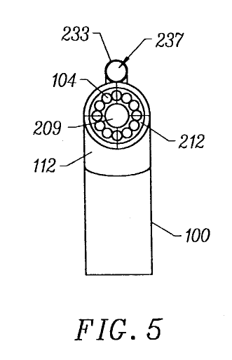

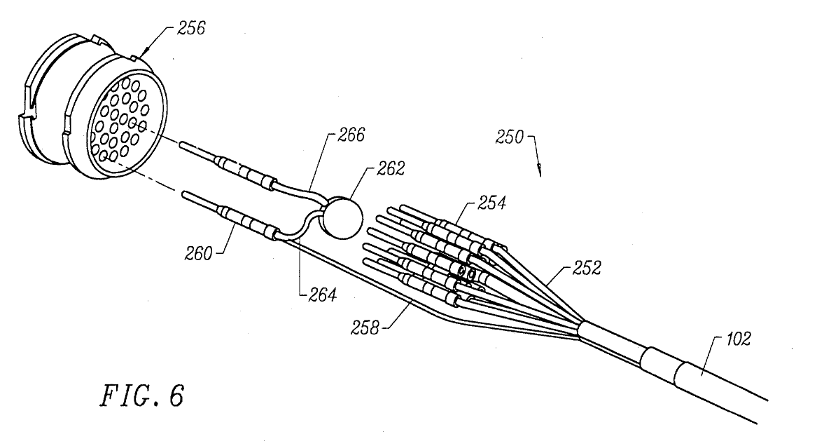

Handle 204 typically comprises a plastic material that is easily molded into a suitable shape for handling by the surgeon. Handle 204 defines an inner cavity (not shown) that houses the electrical connections 250 (FIG. 6), and provides a suitable interface for connection to an electrical connecting cable 22 (see FIG. 1). Electrode support member 102 extends from the distal end of shaft 100 (usually about 1 mm to 20 mm), and provides support for a plurality of electrically isolated active electrodes 104 (see FIG. 5). As shown in FIG. 4, a fluid tube 233 extends through an opening in handle 204, and includes a connector 235 for connection to a fluid supply source, for supplying electrically conductive fluid to the target site. Depending on the configuration of the distal surface of shaft 100, fluid tube 233 may extend through a single lumen (not shown) in shaft 100, or it may be coupled to a plurality of lumens (also not shown) that extend through shaft 100 to a plurality of openings at its distal end. In the representative embodiment, fluid tube 239 is a plastic tubing that extends along the exterior of shaft 100 to a point just distal of return electrode 112 (see FIG. 5). In this embodiment, the fluid is directed through an opening 237 past return electrode 112 to the active electrodes 104. Probe 20 may also include a valve 17 (FIG. 1) or equivalent structure for controlling the flow rate of the electrically conductive fluid to the target site.

As shown in FIG. 4, the distal portion of shaft 100 may be bent to improve access to the operative site of the tissue being treated. Electrode support member 102 has a substantially planar tissue treatment surface 212 (FIG. 5) that is usually at an angle of about 10 degrees to 90 degrees relative to the longitudinal axis of shaft 100, preferably about 30 degrees to 60 degrees, and more preferably about 45 degrees. In alternative embodiments, the distal portion of shaft 100 comprises a flexible material which can be deflected relative to the longitudinal axis of the shaft. Such deflection may be selectively induced by mechanical tension of a pull wire, for example, or by a shape memory wire that expands or contracts by externally applied temperature changes. A more complete description of this embodiment can be found in U.S. Pat. No. 5,697,909, the complete disclosure of which is incorporated herein by reference. Alternatively, shaft 100 of the present invention may be bent by the physician to the appropriate angle using a conventional bending tool or the like.

In the embodiment shown in FIGS. 4 to 6, probe 20 includes a return electrode 112 for completing the current path between active electrodes 104 and a high frequency power supply 28 (see FIG. 1). As shown, return electrode 112 preferably comprises an exposed portion of shaft 100 shaped as an annular conductive band near the distal end of shaft 100 slightly proximal to tissue treatment surface 212 of electrode support member 102, typically about 0.5 mm to 10 mm and more preferably about 1 mm to 10 mm. Return electrode 112 or shaft 100 is coupled to a connector 258 (FIG. 6) that extends to the proximal end of probe 10, where it is suitably connected to power supply 28 (FIG. 1).

As shown in FIG. 4, return electrode 112 is not directly connected to active electrodes 104. To complete a current path so that active electrodes 104 are electrically connected to return electrode 112, an electrically conductive fluid (e.g., isotonic saline) is positioned, or caused to flow, therebetween. In the representative embodiment, the electrically conductive fluid is delivered through fluid tube 233 to opening 237, as described above. Alternatively, the conductive fluid may be delivered by a fluid delivery element (not shown) that is separate from probe 20. In arthroscopic surgery, for example, the joint cavity will be flooded with isotonic saline and the probe 20 will be introduced into this flooded joint cavity. Electrically conductive fluid can be continually resupplied to maintain the conduction path between return electrode 112 and active electrodes 104. In other embodiments, the distal portion of probe 20 may be dipped into a source of electrically conductive fluid, such as a gel or isotonic saline, prior to positioning the probe distal portion at the target site. Applicant has found that the surface tension of the fluid and/or the viscous nature of a gel allows the conductive fluid to remain around the active and return electrodes for long enough to complete its function according to the present invention, as described below. Alternatively, the conductive fluid, such as a gel, may be applied directly to the target site.

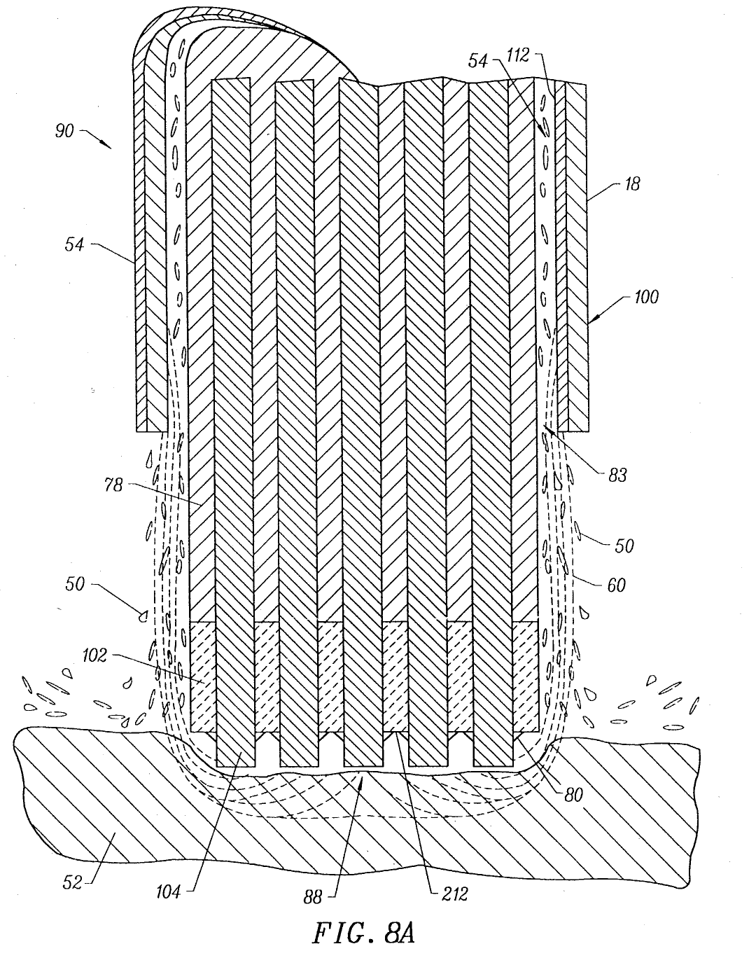

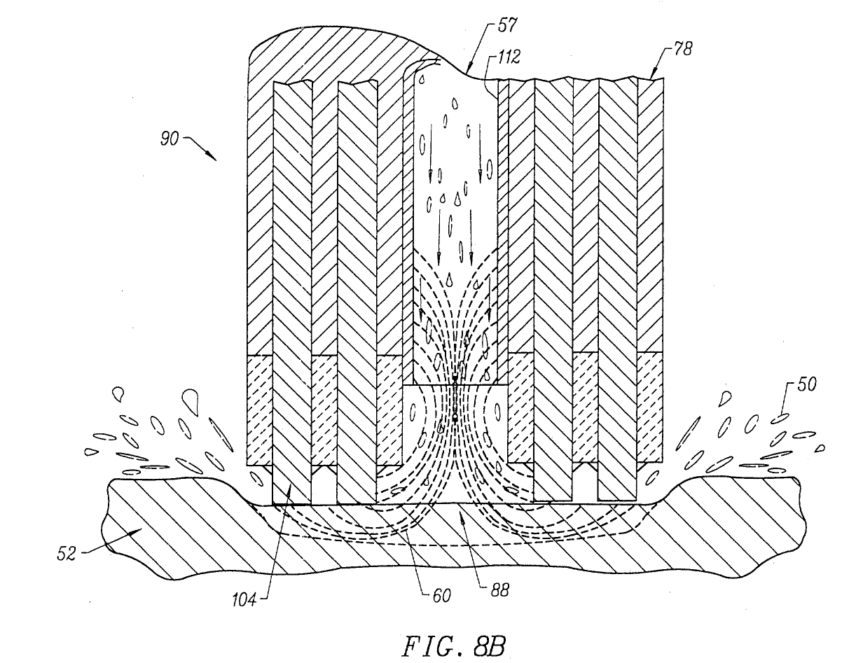

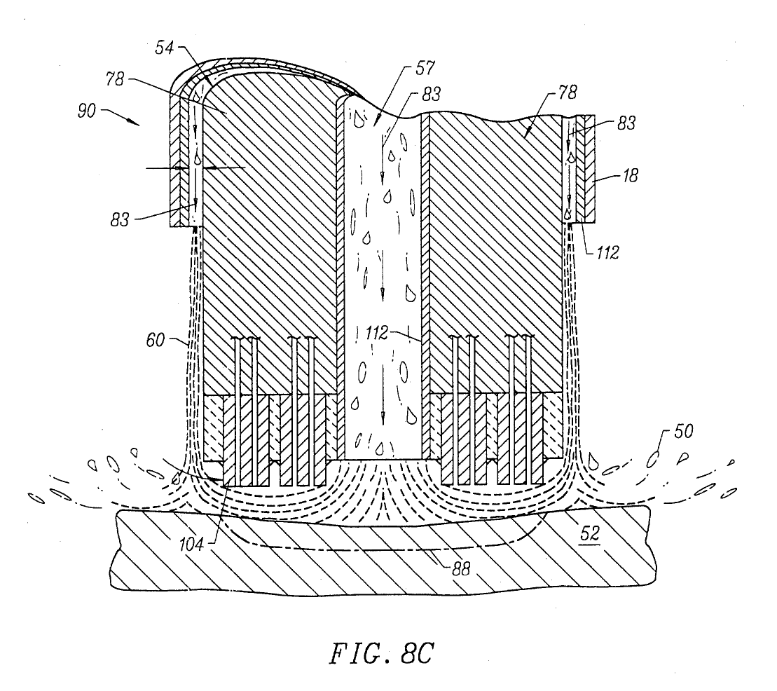

In alternative embodiments, the fluid path may be formed in probe 20 by, for example, an inner lumen or an annular gap between the return electrode and a tubular support member within shaft 100 (see FIGS. 8A and 8B). This annular gap may be formed near the perimeter of the shaft 100 such that the electrically conductive fluid tends to flow radially inward towards the target site, or it may be formed towards the center of shaft 100 so that the fluid flows radially outward. In both of these embodiments, a fluid source (e.g., a bag of fluid elevated above the surgical site or having a pumping device), is coupled to probe 20 via a fluid supply tube (not shown) that may or may not have a controllable valve. A more complete description of an electrosurgical probe incorporating one or more fluid lumen(s) can be found in U.S. Pat. No. 5,697,281, the complete disclosure of which is incorporated herein by reference.

Referring to FIG. 5, the electrically isolated active electrodes 104 are spaced apart over tissue treatment surface 212 of electrode support member 102. The tissue treatment surface and individual active electrodes 104 will usually have dimensions within the ranges set forth above. In the representative embodiment, the tissue treatment surface 212 has a circular cross-sectional shape with a diameter in the range of 1 mm to 20 mm. The individual active electrodes 104 preferably extend from tissue treatment surface 212 by a distance of about 0.1 mm to 4 mm, usually about 0.2 mm to 2 mm. Applicant has found that this configuration increases the high electric field intensities and associated current densities around active electrodes 104 to facilitate the ablation and shrinkage of tissue as described in detail above.

In the embodiment of FIGS. 4 and 5, the probe includes a single, larger opening 209 in the center of tissue treatment surface 212, and a plurality of active electrodes (e.g., about 3-15) around the perimeter of surface 212 (see FIG. 5). Alternatively, the probe may include a single, annular, or partially annular, active electrode at the perimeter of the tissue treatment surface. The central opening 209 is coupled to a suction lumen (not shown) within shaft 100 and a suction tube 211 (FIG. 4) for aspirating tissue, fluids and/or gases from the target site. In this embodiment, the electrically conductive fluid generally flows radially inward past active electrodes 104 and then back through the opening 209. Aspirating the electrically conductive fluid during surgery allows the surgeon to see the target site, and it prevents the fluid from flowing into the patient's body.

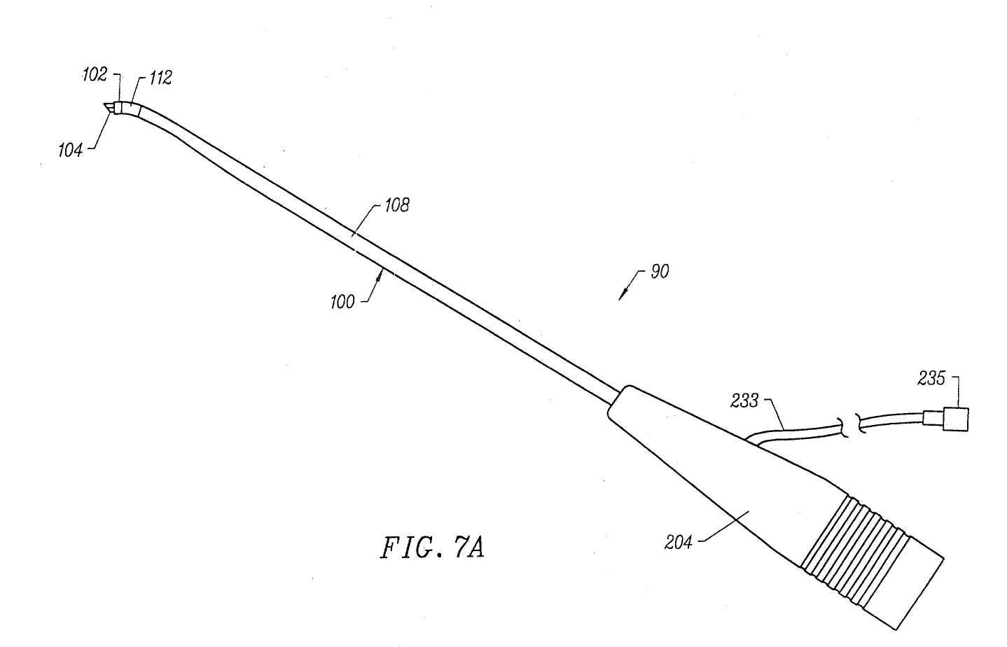

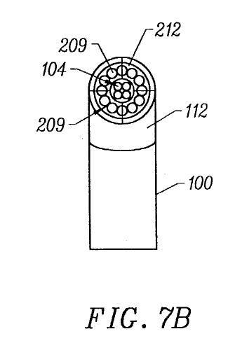

Of course, it will be recognized that the distal tip of the probe may have a variety of different configurations. For example, the probe may include a plurality of openings 209 around the outer perimeter of tissue treatment surface 212 (see FIG. 7B). In this embodiment, the active electrodes 104 extend distally from the center of tissue treatment surface 212 such that they are located radially inward from openings 209. The openings are suitably coupled to fluid tube 233 for delivering electrically conductive fluid to the target site, and suction tube 211 for aspirating the fluid after it has completed the conductive path between the return electrode 112 and the active electrodes 104.

FIG. 6 illustrates the electrical connections 250 within handle 204 for coupling active electrodes 104 and return electrode 112 to the power supply 28. As shown, a plurality of wires 252 extend through shaft 100 to couple active electrodes 104 to a plurality of pins 254, which are plugged into a connector block 256 for coupling to a connecting cable 22 (FIG. 1). Similarly, return electrode 112 is coupled to connector block 256 via a wire 258 and a plug 260.

According to the present invention, the probe 20 further includes an identification element that is characteristic of the particular electrode assembly so that the same power supply 28 can be used for different electrosurgical operations. In one embodiment, for example, the probe 20 includes a voltage reduction element or a voltage reduction circuit for reducing the voltage applied between the active electrodes 104 and the return electrode 112. The voltage reduction element serves to reduce the voltage applied by the power supply so that the voltage between the active electrodes and the return electrode is low enough to avoid excessive power dissipation into the electrically conductive medium and/or ablation of the tissue at the target site. In some embodiments, the voltage reduction element allows the power supply 28 to apply two different voltages simultaneously to two different electrodes (see FIG. 15D). In other embodiments, the voltage reduction element primarily allows the electrosurgical probe 20/90 to be compatible with other power supply units (for example, various electrosurgical power supply units manufactured by ArthroCare Corporation, Sunnyvale, Calif.) that are adapted to apply higher voltages for ablation or vaporization of tissue. For thermal heating or coagulation of tissue, for example, the voltage reduction element will serve to reduce a voltage of about 100 volts RMS to 170 volts RMS (which is a setting of 1 or 2 on the ArthroCare Model 970 and 980 (i.e., 2000) Generators (ArthroCare Corporation, Sunnyvale, Calif.)) to about 45 volts RMS to 60 volts RMS, which is a suitable voltage for coagulation of tissue without ablation (e.g., molecular dissociation) of the tissue.

Of course, for some procedures, the probe will typically not require a voltage reduction element. Alternatively, the probe may include a voltage increasing element or circuit, if desired. Alternatively or additionally, the cable 22 that couples the power supply 28 to the probe may be used as a voltage reduction element. The cable has an inherent capacitance that can be used to reduce the power supply voltage if the cable is placed into the electrical circuit between the power supply, the active electrodes and the return electrode. In this embodiment, the cable 22 may be used alone, or in combination with one of the voltage reduction elements discussed above, e.g., a capacitor. Further, it should be noted that the present invention can be used with a power supply that is adapted to apply a voltage within the selected range for treatment of tissue. In this embodiment, a voltage reduction element or circuitry may not be desired.

FIGS. 8A-8C schematically illustrate the distal portion of three different embodiments of probe 90 according to the present invention. As shown in 8A, active electrodes 104 are anchored in electrode support 102. Electrode support 102 may comprise a matrix of suitable insulating material (e.g., a silicone rubber, a ceramic, or glass material, such as alumina, zirconia and the like) which could be formed at the time of manufacture in a flat, hemispherical or other shape according to the requirements of a particular procedure. In one embodiment, the support matrix material is alumina (available from Kyocera Industrial Ceramics Corporation, Elkgrove, Ill.). Alumina has the advantages of high thermal conductivity, good electrically insulative properties, high flexural modulus, resistance to carbon tracking, biocompatibility, and high melting point. The support 102 is adhesively joined to a tubular support member 78 that extends most or all of the distance between support 102 and the proximal end of probe 90. Tubular member 78 preferably comprises an electrically insulating material, such as an epoxy or silicone-based material.

In a preferred construction technique, active electrodes 104 extend through pre-formed openings in the support 102 so that they protrude above tissue treatment surface 212 by the desired distance. The electrodes are then bonded to the tissue treatment surface 212 of support 102, typically by an inorganic sealing material 80. Sealing material 80 is selected to provide effective electrical insulation, and good adhesion to both alumina support 102 and the platinum or titanium active electrodes. Sealing material 80 additionally should have a compatible thermal expansion coefficient and a melting point well below that of platinum or titanium and alumina or zirconia, typically being a glass or glass ceramic.

In the embodiment shown in FIG. 8A, return electrode 112 comprises an annular member positioned around the exterior of shaft 100 of probe 90. Return electrode 112 may fully or partially circumscribe tubular support member 78 to form an annular gap 54 therebetween for flow of electrically conductive liquid 50 therethrough, as discussed below. Gap 54 preferably has a width in the range of 0.25 mm to 4 mm. Alternatively, probe may include a plurality of longitudinal ribs between tubular support member 78 and return electrode 112 to form a plurality of fluid lumens extending along the perimeter of shaft 100. In this embodiment, the plurality of lumens will extend to a plurality of openings.

Return electrode 112 is disposed within an electrically insulative jacket 18, which is typically formed as one or more electrically insulative sheaths or coatings, such as polytetrafluoroethylene, polyamide, and the like. The provision of the electrically insulative jacket 18 over return electrode 112 prevents direct electrical contact between return electrode 112 and any adjacent, non-target tissue or body structure. As shown in FIG. 8A, return electrode 112 is not directly connected to active electrodes 104. To complete this current path so that terminals 104 are electrically connected to return electrode 112, electrically conductive liquid 50 (e.g., isotonic saline) is caused to flow along fluid path(s) 83. Fluid path 83 is formed by annular gap 54 between outer return electrode 112 and tubular support member 78. The electrically conductive liquid 50 flowing through fluid path 83 provides a pathway for electrical current flow between active electrodes 104 and return electrode 112, as illustrated by the current flux lines 60 in FIG. 8A. When a voltage is applied between active electrodes 104 and return electrode 112, high electric field intensities will be generated at the distal tips of active electrodes 104 with current flow from active electrodes 104 through the target tissue to return electrode 112, the high electric field intensities causing ablation of tissue 52 in zone 88.

FIG. 8B illustrates another alternative embodiment of electrosurgical probe 90 which has a return electrode 112 positioned within tubular member 78. Return electrode 112 is preferably substantially cylindrical defining an inner lumen 57 for allowing electrically conductive liquid 50 (e.g., isotonic saline) to flow therethrough in electrical contact with return electrode 112. In this embodiment, a voltage difference is applied between active electrodes 104 and return electrode 112 resulting in electrical current flow through the electrically conductive liquid 50 as shown by current flux lines 60. As a result of the applied voltage and concomitant high electric field intensities at the tips of active electrodes 104, tissue 52 becomes ablated or transected in zone 88.

FIG. 8C illustrates another embodiment of probe 90 that is a combination of the embodiments in FIGS. 8A and 8B. As shown, this probe includes both an inner lumen 57 and an outer gap or plurality of outer lumens 54 for flow of electrically conductive fluid. In this embodiment, the return electrode 112 may be positioned within tubular member 78 as in FIG. 8B, outside of tubular member 78 as in FIG. 8A, or in both locations.

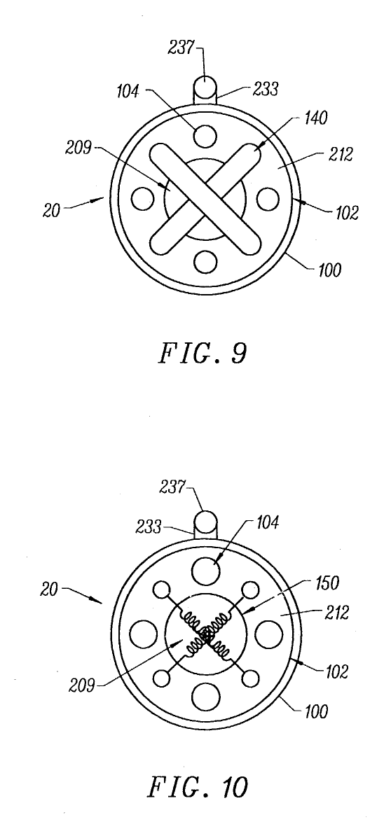

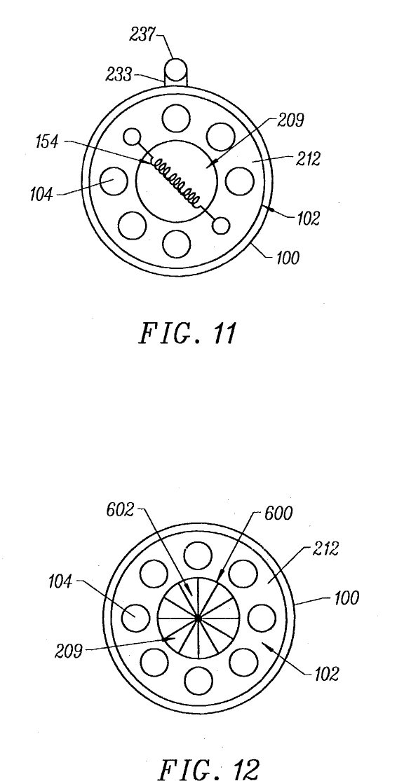

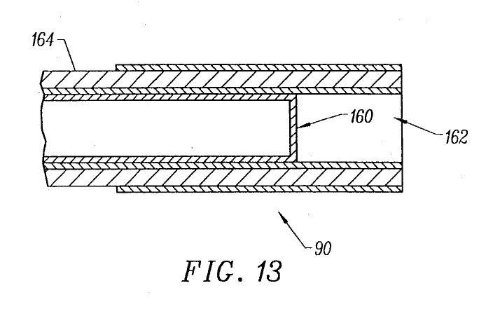

In some embodiments, the probe 20/90 will also include one or more aspiration electrode(s) coupled to the aspiration lumen for inhibiting clogging during aspiration of tissue fragments from the surgical site. As shown in FIG. 9, one or more of the active electrodes 104 may comprise loop electrodes 140 that extend across distal opening 209 of the suction lumen within shaft 100. In the representative embodiment, two of the active electrodes 104 comprise loop electrodes 140 that cross over the distal opening 209. Of course, it will be recognized that a variety of different configurations are possible, such as a single loop electrode, or multiple loop electrodes having different configurations than shown. In addition, the electrodes may have shapes other than loops, such as the coiled configurations shown in FIGS. 10 and 11. Alternatively, the electrodes may be formed within the suction lumen proximal to the distal opening 209, as shown in FIG. 13. The main function of loop electrodes 140 is to ablate portions of tissue that are drawn into the suction lumen to prevent clogging of the lumen.

In some embodiments, loop electrodes 140 are electrically isolated from the other active electrodes 104. In other embodiments, the loop electrodes 140 and active electrodes 104 may be electrically connected to each other such that both are activated together. Loop electrodes 140 may or may not be electrically isolated from each other. Loop electrodes 140 will usually extend only about 0.05 mm to 4 mm, preferably about 0.1 mm to 1 mm, from the tissue treatment surface of electrode support member 102.

Referring now to FIGS. 10 and 11, alternative embodiments for aspiration electrodes will now be described. As shown in FIG. 10, the aspiration electrodes may comprise a pair of coiled electrodes 150 that extend across distal opening 209 of the suction lumen. The larger surface area of the coiled electrodes 150 usually increases the effectiveness of the electrodes 150 in ablating or digesting tissue fragments passing through opening 209. In FIG. 11, the aspiration electrode comprises a single coiled electrode 154 extending across the distal opening 209 of the suction lumen. This single electrode 154 may be sufficient to inhibit clogging of the suction lumen. Alternatively, the aspiration electrodes may be positioned within the suction lumen proximal to the distal opening 209. Preferably, these electrodes are close to opening 209 so that tissue does not clog the opening 209 before it reaches electrode 154. In this embodiment, a separate return electrode 156 (not shown) may be provided within the suction lumen to confine the electric currents therein.

Referring to FIG. 13, another embodiment of the present invention incorporates an aspiration electrode 160 within the aspiration lumen 162 of the probe. As shown, the electrode 160 is positioned just proximal of distal opening 209 so that the tissue fragments are ablated as they enter lumen 162. In the representative embodiment, the aspiration electrode 160 comprises a loop electrode that extends across the aspiration lumen 162. However, it will be recognized that many other configurations are possible. In this embodiment, the return electrode 164 is located on the exterior of the probe as in the previously described embodiments. Alternatively, the return electrode(s) may be located within the aspiration lumen 162 with the aspiration electrode 160. For example, inner insulating coating 163 may be exposed at portions within the lumen 162 to provide a conductive path between this exposed portion of return electrode 164 and the aspiration electrode 160. The latter embodiment has the advantage of confining the electric currents to within the aspiration lumen. In addition, in dry fields in which the conductive fluid is delivered to the target site, it is usually easier to maintain a conductive fluid path between the active and return electrodes in the latter embodiment because the conductive fluid is aspirated through the aspiration lumen 162 along with the tissue fragments.

Referring to FIG. 12, another embodiment of the present invention incorporates a wire mesh electrode 600 extending across the distal portion of aspiration lumen 162. As shown, mesh electrode 600 includes a plurality of openings 602 to allow fluids and tissue fragments to flow through into aspiration lumen 162. The size of the openings 602 will vary depending on a variety of factors. The mesh electrode may be coupled to the distal or proximal surfaces of support member 102. Wire mesh electrode 600 comprises a conductive material, such as titanium, tantalum, steel, stainless steel, tungsten, copper, or gold, and the like. In the representative embodiment, wire mesh electrode 600 comprises a different material, having a different electric potential, than the active electrode(s) 104. In one embodiment, mesh electrode 600 comprises steel, and active electrode(s) comprises tungsten. Applicant has found that a slight variance in the electrochemical potential of mesh electrode 600 and active electrode(s) 104 improves the performance of the device. Of course, it will be recognized that the mesh electrode may be electrically insulated from active electrode(s) as in previous embodiments

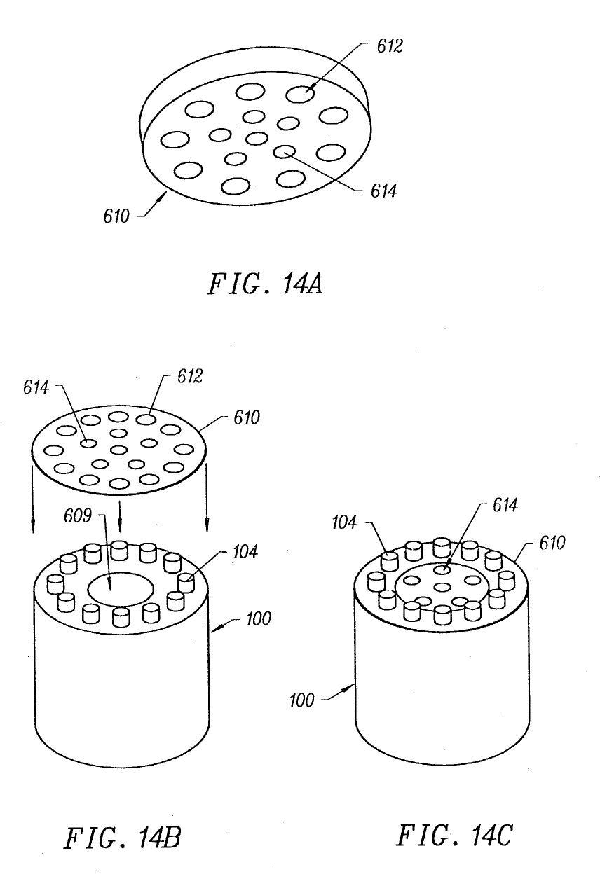

Referring now to FIGS. 14A-14C, an alternative embodiment incorporating a metal screen 610 is illustrated. As shown, metal screen 610 has a plurality of peripheral openings 612 for receiving active electrodes 104, and a plurality of inner openings 614 for allowing aspiration of fluid and tissue through opening 609 of the aspiration lumen. As shown, screen 610 is press fitted over active electrodes 104 and then adhered to shaft 100 of probe 20. Similar to the mesh electrode embodiment, metal screen 610 may comprise a variety of conductive metals, such as titanium, tantalum, steel, stainless steel, tungsten, copper, gold or the like. In the representative embodiment, metal screen 610 is coupled directly to, or integral with, active electrode(s) 104. In this embodiment, the active electrode(s) 104 and the metal screen 610 are electrically coupled to each other.

Referring to FIG. 15A, probe 350 comprises an electrically conductive shaft 352, a handle 354 coupled to the proximal end of shaft 352 and an electrically insulating support member 356 at the distal end of shaft 352. Probe 350 further includes a shrink wrapped insulating sleeve 358 over shaft 352, and an exposed portion of shaft 352 that functions as the return electrode 360. In the representative embodiment, probe 350 comprises a plurality of active electrodes 362 extending from the distal end of support member 356. As shown, return electrode 360 is spaced a further distance from active electrodes 362 than in the embodiments described above. In this embodiment, the return electrode 360 is spaced a distance of about 2.0 mm to 50 mm, preferably about 5 mm to 25 mm. In addition, return electrode 360 has a larger exposed surface area than in previous embodiments, having a length in the range of about 2.0 mm to 40 mm, preferably about 5 mm to 20 mm. Accordingly, electric current passing from active electrodes 362 to return electrode 360 will follow a current flow path 370 that is further away from shaft 352 than in the previous embodiments. In some applications, this current flow path 370 results in a deeper current penetration into the surrounding tissue with the same voltage level, and thus increased thermal heating of the tissue. As discussed above, this increased thermal heating may have advantages in some applications of treating disc or other spinal defects or disorders. Typically, it is desired to achieve a tissue temperature in the range of about 60° C. to 100° C. to a depth of about 0.2 mm to 5 mm, usually about 1 mm to 2 mm. The voltage required for this thermal heating will partly depend on the electrode configurations, the conductivity of the tissue and the area immediately surrounding the electrodes, the time period in which the voltage is applied, and the depth of tissue heating desired. With the electrode configurations described in FIGS. 15A-15D, the voltage level for thermal heating will usually be in the range of about 20 volts RMS to 300 volts RMS, and preferably about 60 volts RMS to 200 volts RMS. The peak-to-peak voltages for thermal heating with a square wave form having a crest factor of about 2 are typically in the range of about 40 to 600 volts peak-to-peak, preferably about 120 to 400 volts peak-to-peak. The higher the voltage is within this range, the less time required. If the voltage is too high, however, the surface tissue may be vaporized, debulked or ablated, which is undesirable in certain procedures.

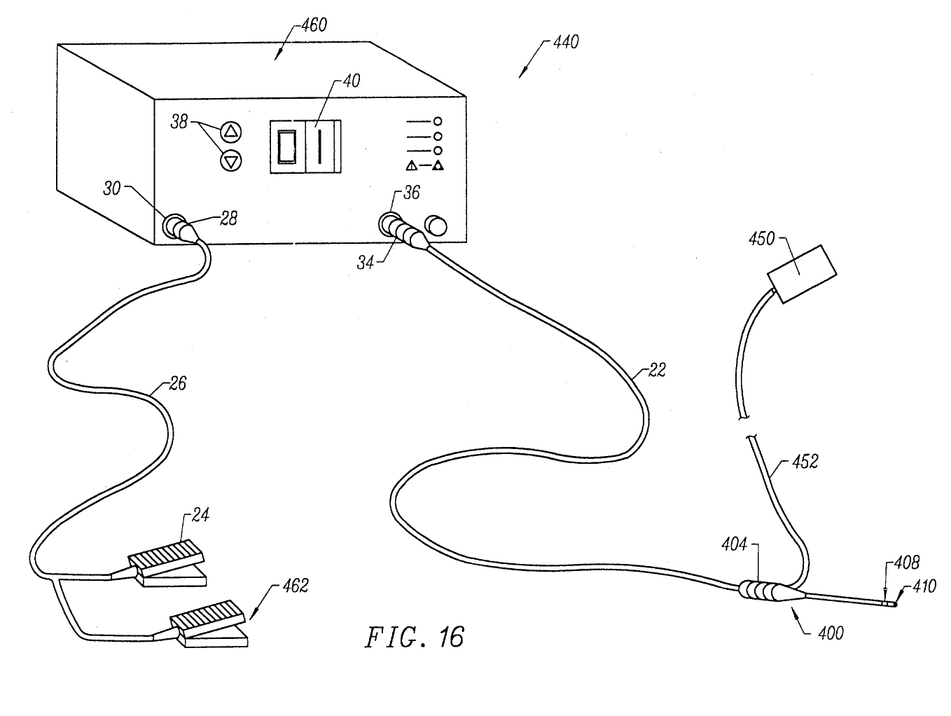

In alternative embodiments, the electrosurgical system used in conjunction with probe 350 may include a dispersive return electrode 450 (see FIG. 16) which allows for switching between bipolar and monopolar modes. In this embodiment, the system will switch between an ablation mode, where the dispersive pad 450 is deactivated and voltage is applied between active and return electrodes 362, 360, and a sub-ablation or thermal heating mode, where the active electrode(s) 362 are deactivated and voltage is applied between the dispersive pad 450 and the return electrode 360. In the sub-ablation mode, a lower voltage is typically applied and the return electrode 360 functions as the active electrode to provide thermal heating and/or coagulation of tissue surrounding return electrode 360.

FIG. 15B illustrates yet another embodiment of the present invention. As shown, electrosurgical probe 350 comprises an electrode assembly 372 having one or more active electrode(s) 362 and a proximally spaced return electrode 360 as in previous embodiments. Return electrode 360 is typically spaced about 0.5 mm to 25 mm, preferably 1.0 mm to 5.0 mm from the active electrode(s) 362, and has an exposed length of about 1 mm to 20 mm. In addition, electrode assembly 372 includes two additional electrodes 374, 376 spaced axially on either side of return electrode 360. Electrodes 374, 376 are typically spaced about 0.5 mm to 25 mm, preferably about 1 mm to 5 mm from return electrode 360. In the representative embodiment, the additional electrodes 374, 376 are exposed portions of shaft 352, and the return electrode 360 is electrically insulated from shaft 352 such that a voltage difference may be applied between electrodes 374, 376 and electrode 360. In this embodiment, probe 350 may be used in at least two different modes, an ablation mode and a sub-ablation or thermal heating mode. In the ablation mode, voltage is applied between active electrode(s) 362 and return electrode 360 in the presence of electrically conductive fluid, as described above. In the ablation mode, electrodes 374, 376 are deactivated. In the thermal heating or coagulation mode, active electrode(s) 362 are deactivated and a voltage difference is applied between electrodes 374, 376 and electrode 360 such that a high frequency current 370 flows therebetween, as shown in FIG. 15B. In the thermal heating mode, a lower voltage is typically applied, such that the applied voltage is below the threshold for plasma formation and ablation, but sufficient to cause some thermal effect on the tissue immediately surrounding the electrodes without vaporizing or otherwise debulking this tissue, so that the current 370 provides thermal heating and/or coagulation of tissue surrounding electrodes 360, 372, 374.

FIG. 15C illustrates another embodiment of probe 350 incorporating an electrode assembly 372 having one or more active electrode(s) 362 and a proximally spaced return electrode 360 as in previous embodiments. Return electrode 360 is typically spaced about 0.5 mm to 25 mm, preferably 1.0 mm to 5.0 mm from the active electrode(s) 362, and has an exposed length of about 1 mm to 20 mm. In addition, electrode assembly 372 includes a second active electrode 380 separated from return electrode 360 by an electrically insulating spacer 382. In this embodiment, handle 354 includes a switch 384 for toggling probe 350 between at least two different modes, an ablation mode and a sub-ablation or thermal heating mode. In the ablation mode, voltage is applied between active electrode(s) 362 and return electrode 360 in the presence of electrically conductive fluid, as described above. In the ablation mode, electrode 380 is deactivated. In the thermal heating or coagulation mode, active electrode(s) 362 may be deactivated and a voltage difference is applied between electrode 380 and electrode 360 such that a high frequency current 370 flows therebetween. Alternatively, active electrode(s) 362 may not be deactivated as the higher resistance of the smaller electrodes may automatically send the electric current to electrode 380 without having to physically decouple electrode(s) 362 from the circuit. In the thermal heating mode, a lower voltage is typically applied below the threshold for plasma formation and ablation, but sufficient to cause some thermal effect on the tissue immediately surrounding the electrodes without vaporizing or otherwise debulking this tissue so that the current 370 provides thermal heating and/or coagulation of tissue surrounding electrodes 360, 380.

Of course, it will be recognized that a variety of other embodiments may be used to accomplish similar functions as the embodiments described above. For example, electrosurgical probe 350 may include a plurality of helical bands formed around shaft 352, with one or more of the helical bands having an electrode coupled to the portion of the band such that one or more electrodes are formed on shaft 352 spaced axially from each other.

FIG. 15D illustrates another embodiment of the invention designed for channeling through tissue and creating lesions therein. As shown, probe 350 is similar to the probe in FIG. 15C having a return electrode 360 and a third, coagulation electrode 380 spaced proximally from the return electrode 360. In this embodiment, active electrode 362 comprises a single electrode wire extending distally from insulating support member 356. Of course, the active electrode 362 may have a variety of configurations to increase the current densities on its surfaces, e.g., a conical shape tapering to a distal point, a hollow cylinder, a loop electrode, and the like. In the representative embodiment, support member 356 and spacer 382 are constructed of an electrically insulating material, such as a ceramic, a glass, a silicone rubber, and the like. The proximal insulating spacer 382 may alternatively comprise a more conventional organic insulating material.

In one embodiment, probe 350 of FIG. 15D does not include a switching element, wherein all three electrodes are activated when the power supply is activated. The return electrode 360 has an opposite polarity from the active and coagulation electrodes 362, 380 such that current 370 flows from the latter electrodes to the return electrode 360 as shown. In one embodiment, the electrosurgical system includes a voltage reduction element, or a voltage reduction circuit, for reducing the voltage applied between the coagulation electrode 380 and return electrode 360. The voltage reduction element allows the power supply 28 to, in effect, apply two different voltages simultaneously to two different electrodes. Thus, for channeling through tissue, the operator may apply a voltage sufficient to provide ablation of the tissue at the tip of the probe (i.e., tissue adjacent to the active electrode 362). At the same time, the voltage applied to the coagulation electrode 380 will be insufficient to ablate tissue. For thermal heating or coagulation of tissue, for example, the voltage reduction element will serve to reduce a voltage in the range of about 100-300 volts RMS to about 45-90 volts RMS, the latter generally representing a suitable voltage range for coagulation of tissue without ablation of the tissue.

In the representative embodiment, the voltage reduction element comprises a pair of capacitors forming a bridge divider (not shown) coupled to the power supply and coagulation electrode 380. The capacitor usually has a capacitance of about 200 pF to 500 pF (at 500 volts), and preferably about 300 pF to 350 pF (at 500 volts). Of course, the capacitors may be located in other places within the system, such as in, or distributed along the length of, the cable, the generator, the connector, etc. In addition, it will be recognized that other voltage reduction elements, such as diodes, transistors, inductors, resistors, capacitors or combinations thereof, may be used in conjunction with the present invention. For example, the probe 350 may include a coded resistor (not shown) that is constructed to lower the voltage applied between the return and coagulation electrodes 360, 380. In addition, electrical circuits may be employed for this purpose.

Of course, for some procedures, the probe will typically not require a voltage reduction element. Alternatively, the probe may include a voltage increasing element or circuit, if desired. Alternatively or additionally, the cable 22 that couples the power supply 28 to the probe 90 may be used as a voltage reduction element. The cable has an inherent capacitance that can be used to reduce the power supply voltage if the cable is placed into the electrical circuit between the power supply, the active electrodes and the return electrode. In this embodiment, the cable 22 may be used alone, or in combination with one of the voltage reduction elements discussed above, e.g., a capacitor. Further, it should be noted that the present invention can be used with a power supply that is adapted to apply two different voltages within the selected range for treatment of tissue. In this embodiment, a voltage reduction element or circuitry may not be desired.

In one specific embodiment, the probe 350 is manufactured by first inserting an electrode wire (active electrode 362) through a ceramic tube (insulating support member 356) such that a distal portion of the wire extends through the distal portion of the tube, and bonding the wire to the tube, typically with an appropriate epoxy. A stainless steel tube (return electrode 360) is then placed over the proximal portion of the ceramic tube, and a wire (e.g., nickel wire) is bonded, typically by spot welding, to the inside surface of the stainless steel tube. The stainless steel tube is coupled to the ceramic tube by epoxy, and the device is cured in an oven or other suitable heat source. A second ceramic tube (insulating spacer member 382) is then placed inside the proximal portion of the stainless steel tube, and bonded in a similar manner. The shaft 352 is then bonded to the proximal portion of the second ceramic tube, and insulating sleeve 358 (e.g. polyimide) is wrapped around shaft 352 such that only a distal portion of the shaft is exposed (i.e., coagulation electrode 380). The nickel wire connection will extend through the center of shaft 352 to connect return electrode 360 to the power supply. The active electrode 362 may form a distal portion of shaft 352, or it may also have a connector extending through shaft 352 to the power supply.

In use, the physician positions active electrode 362 in at least close proximity to the tissue to be treated. The power supply is activated to provide an ablation voltage between active and return electrodes 362, 360 and a coagulation or thermal heating voltage between coagulation and return electrodes 380, 360. An electrically conductive fluid can then be provided around active electrode 362, and in the junction between the active and return electrodes 360, 362 to provide a current flow path therebetween. This may be accomplished in a variety of manners, as discussed above. The active electrode 362 may be advanced into the void formed by the ablation of tissue to form a channel in or through the target tissue. During ablation, the electric current between the coagulation and return electrode is typically insufficient to cause any damage to the surface of the tissue as these electrodes pass through the tissue surface into the channel created by active electrode 362. Once the physician has formed a suitable void, hole, or channel in the target tissue,, he or she will cease advancement of the active electrode, and will either hold the instrument in place for approximately 5 seconds to 30 seconds, or can immediately remove the distal tip of the instrument from the channel (see detailed discussion of this below). In either event, when the active electrode is no longer advancing, it will eventually stop ablating tissue.

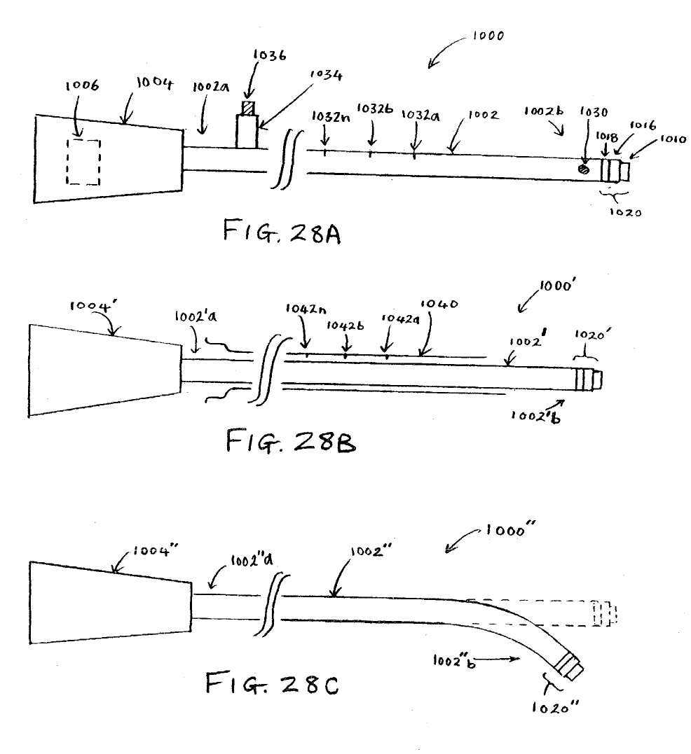

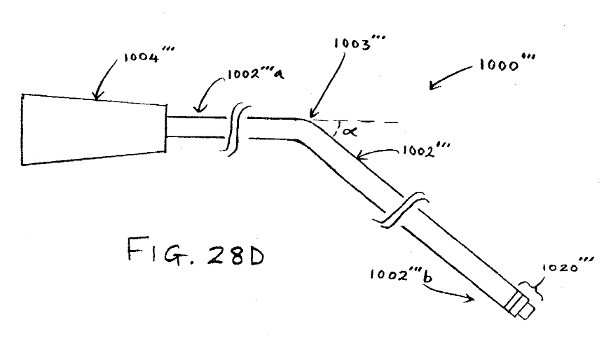

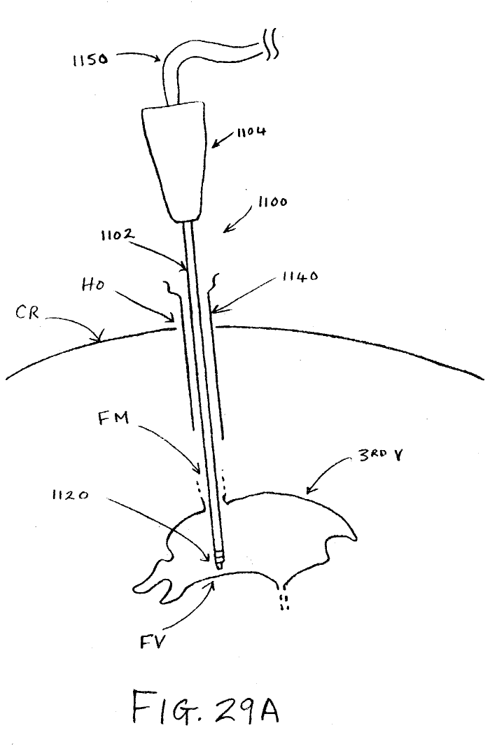

Prior to entering the channel formed by the active electrode 362, an open circuit exists between return and coagulation electrodes 360, 380. Once coagulation electrode 380 enters this channel, electric current will flow from coagulation electrode 380, through the tissue surrounding the channel, to return electrode 360. This electric current will heat the tissue immediately surrounding the channel to coagulate any severed vessels at the surface of the channel. If the physician desires, the instrument may be held within the channel for a period of time to create a lesion around the channel, as discussed in more detail below. Although FIG. 15D shows a bend near the distal tip of the shaft, in alternative embodiments, the shaft may be essentially linear, or may include a bend at other regions of the shaft (see, e.g., FIGS. 28A-C and 28D, respectively).

FIG. 16 illustrates yet another embodiment of an electrosurgical system 440 incorporating a dispersive return pad 450 attached to the electrosurgical probe 400. In this embodiment, the invention functions in the bipolar mode as described above. In addition, the system 440 may function in a monopolar mode in which a high frequency voltage difference is applied between the active electrode(s) 410, and the dispersive return pad 450. In the exemplary embodiment, the pad 450 and the probe 400 are coupled together, and are both disposable, single-use items. The pad 450 includes an electrical connector 452 that extends into handle 404 of probe 400 for direct connection to the power supply. Of course, the invention would also be operable with a standard return pad that connects directly to the power supply. In this embodiment, the power supply 460 will include a switch, e.g., a foot pedal 462, for switching between the monopolar and bipolar modes. In the bipolar mode, the return path on the power supply is coupled to return electrode 408 on probe 400, as described above. In the monopolar mode, the return path on the power supply is coupled to connector 452 of pad 450, active electrode(s) 410 are decoupled from the electrical circuit, and return electrode 408 functions as the active electrode. This allows the surgeon to switch between bipolar and monopolar modes during, or prior to, the surgical procedure. In some cases, it may be desirable to operate in the monopolar mode to provide deeper current penetration and, thus, a greater thermal heating of the tissue surrounding the return electrodes. In other cases, such as ablation of tissue, the bipolar modality may be preferable to limit the current penetration to the tissue.

In one configuration, the dispersive return pad 450 is adapted for coupling to an external surface of the patient in a region substantially close to the target site. For example, during the treatment of tissue in the head and neck, the dispersive return pad is designed and constructed for placement in or around the patient's shoulder, upper back or upper chest region. This design limits the current path through the patient's body to the head and neck area, which minimizes the damage that may be generated by unwanted current paths in the patient's body, particularly by limiting current flow through the patient's heart. The return pad is also designed to minimize the current densities at the pad, to thereby minimize patient skin burns in the region where the pad is attached.

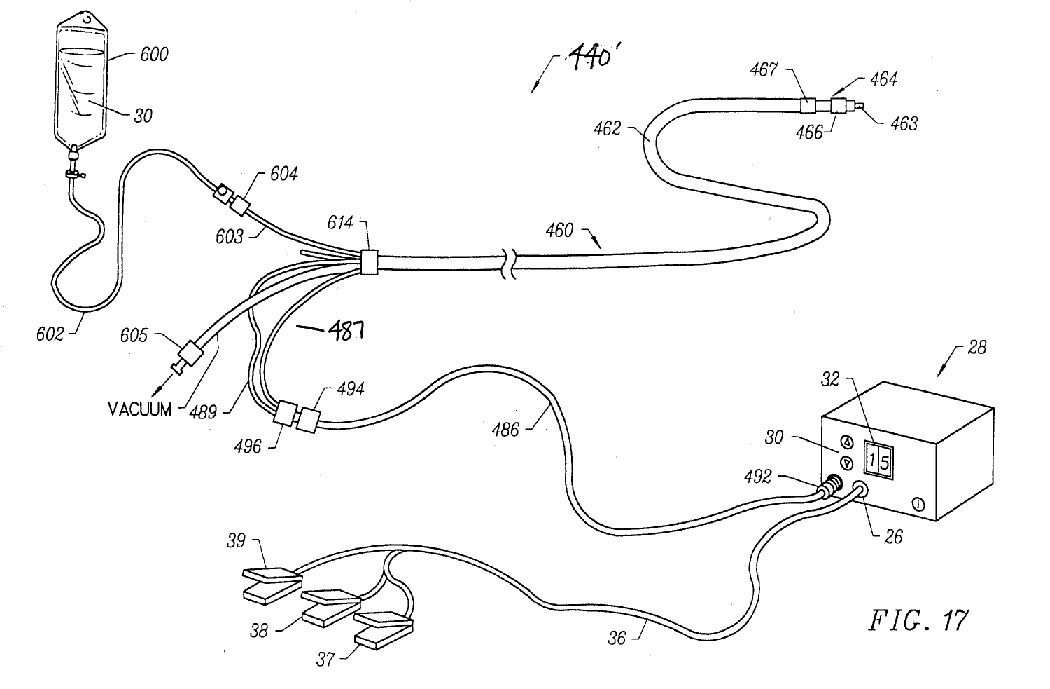

Referring to FIG. 17, an electrosurgical system according to the present invention may also be configured as a catheter system 440′. As shown in FIG. 17, a catheter system 440′ generally comprises an electrosurgical catheter 460 connected to a power supply 28 by an interconnecting cable 486 for providing high frequency voltage to a target tissue, and an irrigant reservoir or fluid source 600 for providing an electrically conductive fluid to the target site. Catheter 460 generally comprises an elongate, flexible shaft body 462 including a tissue removing or ablating region 464 at the distal end of body 462. The proximal portion of catheter 460 includes a multi-lumen fitment 614 which provides for interconnections between lumens and electrical leads within catheter 460 and conduits and cables proximal to fitment 614. By way of example, a catheter electrical connector 496 is removably connected to a distal cable connector 494 which, in turn, is removably connectable to power supply 28 through connector 492. One or more electrically conducting lead wires (not shown) within catheter 460 extend between one or more active electrodes 463 and a coagulation electrode 467 at tissue ablating region 464 and one or more corresponding electrical terminals (also not shown) in catheter connector 496 via active electrode cable branch 487. Similarly, a return electrode 466 at tissue ablating region 464 is coupled to a return electrode cable branch 489 of catheter connector 496 by lead wires (not shown). Of course, a single cable branch (not shown) may be used for both active and return electrodes.

Catheter body 462 may include reinforcing fibers or braids (not shown) in the walls of at least the distal ablation region 464 of body 462 to provide responsive torque control for rotation of active electrodes during tissue engagement. This rigid portion of the catheter body 462 preferably extends only about 7 mm to 10 mm while the remainder of the catheter body 462 is flexible to provide good trackability during advancement and positioning of the electrodes adjacent target tissue. In some embodiments, catheter 460 may be advanced towards the target tissue via a rigid or flexible endoscope (not shown).

In some embodiments, electrically conductive fluid 30 is provided to tissue ablation region 464 of catheter 460 via a lumen (also not shown in FIG. 17) within catheter 460. Fluid is supplied to the lumen from the fluid source via a fluid supply line 602 and a conduit 603, which is coupled to the inner catheter lumen at multi-lumen fitment 614. The source of conductive fluid (e.g., isotonic saline) may be an irrigant pump system (not shown) or a gravity-driven supply, such as an irrigant reservoir 600 positioned several feet above the level of the patient and tissue ablating region 464. A control valve 604 may be positioned at the interface of fluid supply line 602 and conduit 603 to allow manual control of the flow rate of electrically conductive fluid 30. Alternatively, a metering pump or flow regulator may be used to precisely control the flow rate of the conductive fluid. System 440′ can further include an aspiration or vacuum system (not shown) to aspirate liquids and gases from the target site. The aspiration system will usually comprise a source of vacuum coupled to fitment 614 by an aspiration connector 605. The present invention is particularly useful in microendoscopic procedures, e.g., for ablating, coagulating, or otherwise modifying a target tissue in or around the central nervous system.

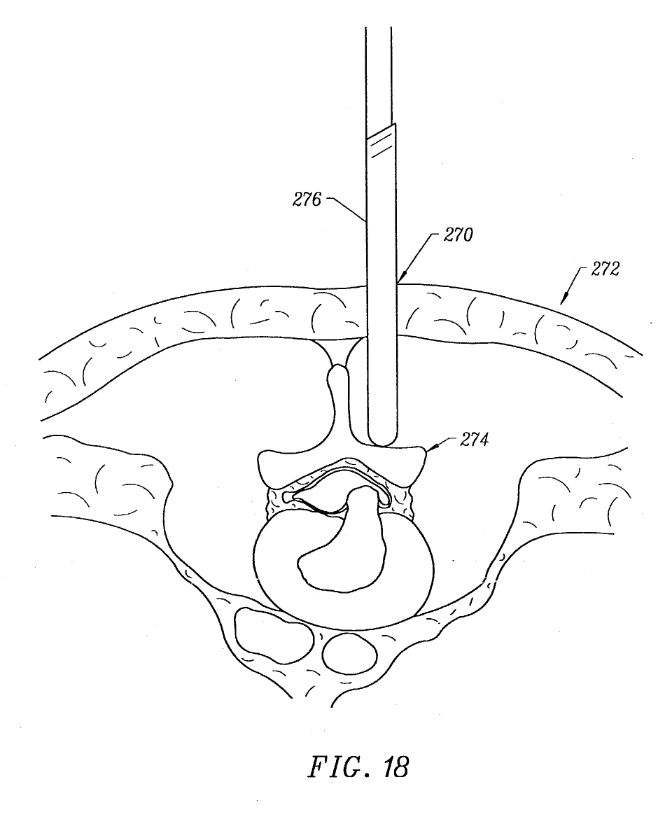

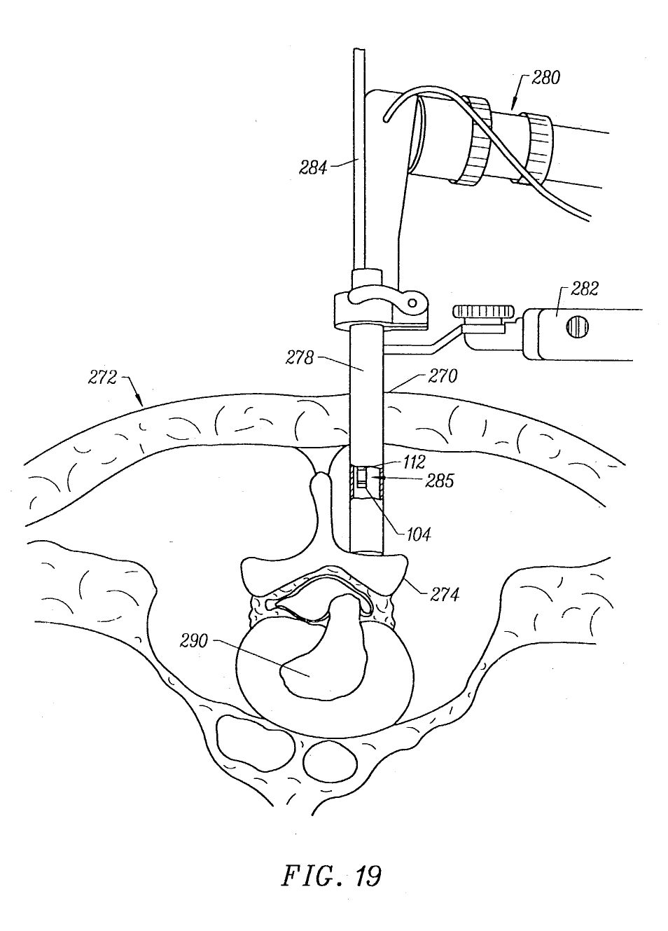

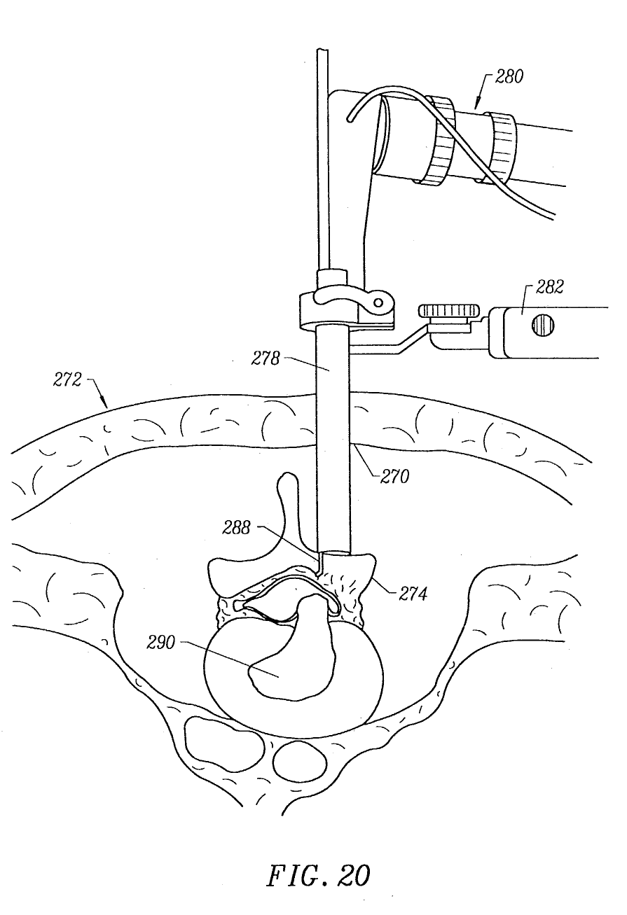

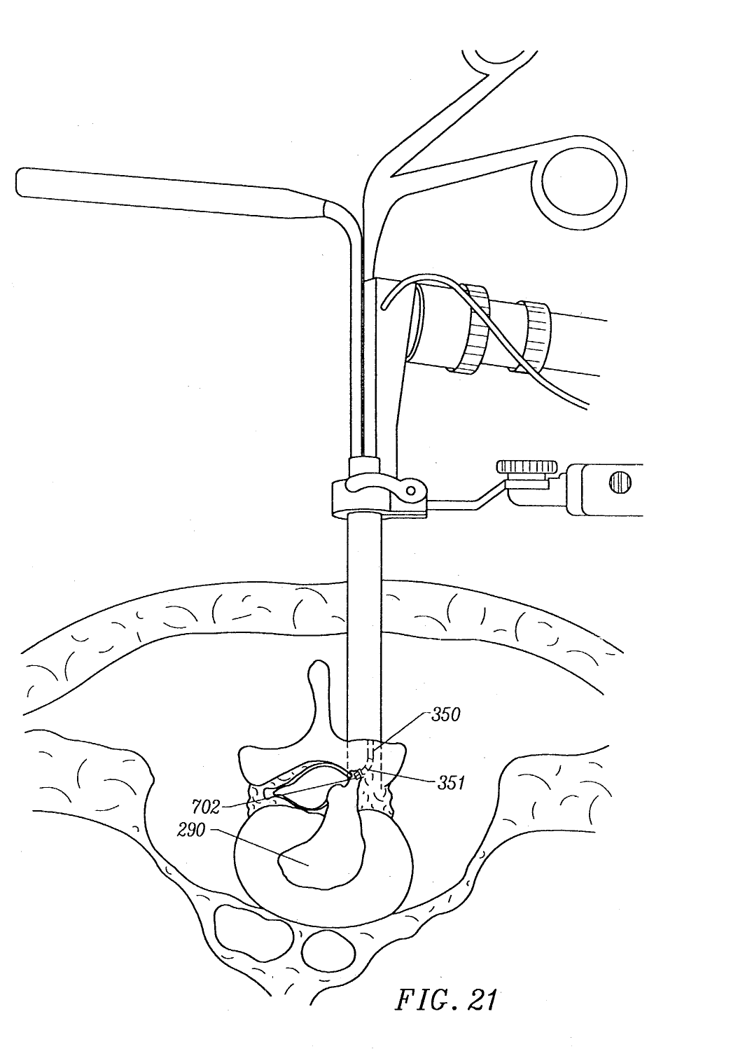

FIGS. 18-21 and 23 each schematically represent a section through a vertebra and vertebral disc, the vertebra or the disc being accessed by an electrosurgical instrument of the invention. As shown in FIGS. 18-23, a percutaneous penetration 270 is made in the patients' back 272 so that the superior lamina 274 can be accessed. Typically, a small needle (not shown) is used initially to localize the disc space level, and a guidewire (not shown) is inserted and advanced under lateral fluoroscopy to the inferior edge of the lamina 274. Sequential cannulated dilators 276 are inserted over the guide wire and each other to provide a hole from the incision 220 to the lamina 274. The first dilator may be used to “palpate” the lamina 274, assuring proper location of its tip between the spinous process and facet complex just above the inferior edge of the lamina 274. As shown in FIGS. 19 and 20 a tubular retractor 278 is then passed over the largest dilator down to the lamina 274. The dilators 276 are removed, establishing an operating corridor within the tubular retractor 278.

As shown in FIG. 19, an endoscope 280 is then inserted into the tubular retractor 278 and a ring clamp 282 is used to secure the endoscope 280. Typically, the formation of the operating corridor within retractor 278 requires the removal of soft tissue, muscle or other types of tissue that were forced into this corridor as the dilators 276 and retractor 278 were advanced down to the lamina 274. In prior art methods, this tissue is usually removed with mechanical instruments, such as pituitary rongeurs, curettes, graspers, cutters, drills, microdebriders, and the like. Unfortunately, these mechanical instruments greatly lengthen and increase the complexity of the procedure. In addition, these prior art instruments sever blood vessels within this tissue, usually causing profuse bleeding that obstructs the surgeon's view of the target site.

According to another aspect of the present invention, an electrosurgical probe or catheter 284 as described above is introduced into the operating corridor within the retractor 278 to remove the soft tissue, muscle and other obstructions from this corridor so that the surgeon can easily access and visualize the lamina 274. Once the surgeon has introduced the probe 284, electrically conductive fluid 285 can be delivered through tube 233 and opening 237 (see FIG. 2) to the tissue. The fluid flows past the return electrode 112 to the active electrodes 104 at the distal end of the probe shaft. The rate of fluid flow is controlled with valve 17 (FIG. 1) such that the zone between the tissue and electrode support 102 is constantly immersed in fluid 285. The power supply 28 is then turned on and adjusted such that a high frequency voltage difference is applied between active electrodes 104 and return electrode 112. The electrically conductive fluid provides the conduction path (see current flux lines) between active electrodes 104 and the return electrode 112.

The high frequency voltage is sufficient to convert the electrically conductive fluid (not shown) between the target tissue and active electrode(s) 104 into an ionized vapor layer or plasma (not shown). As a result of the applied voltage difference between active electrode(s) 104 and the target tissue (i.e., the voltage gradient across the plasma layer), charged particles in the plasma (e.g., electrons) cause molecular dissociation or disintegration of tissue components. This molecular dissociation is accompanied by the volumetric removal of tissue and the production of low molecular weight gases, such as oxygen, nitrogen, carbon dioxide, hydrogen and methane.

During the ablation process of the invention, ablation by-products, e.g., gases, may be aspirated through opening 209 and suction tube 211 to a vacuum source. In addition, excess electrically conductive fluid, or other fluids (e.g., blood) may be aspirated from the operating corridor to facilitate the surgeon's view. During ablation of the tissue, the residual heat generated by the current flux lines (typically less than 150° C.), will usually be sufficient to coagulate any severed blood vessels at the site. If not, the surgeon may switch the power supply 28 into the coagulation mode by lowering the voltage to a level below the threshold for fluid vaporization, as discussed above. This simultaneous hemostasis results in less bleeding and facilitates the surgeon's ability to perform the procedure.

Another advantage of the present invention is the ability to precisely ablate soft tissue without causing necrosis or thermal damage to the underlying and surrounding tissues, nerves or bone. In addition, the voltage can be controlled so that the energy directed to the target site is insufficient to ablate the lamina 274 so that the surgeon can literally clean the tissue off the lamina 274, without ablating or otherwise effecting significant damage to the lamina.

Referring now to FIGS. 20 and 21, once the operating corridor is sufficiently cleared, a laminotomy and medial facetectomy is accomplished either with conventional techniques (e.g., Kerrison punch or a high speed drill) or with the electrosurgical probe 284 as discussed above. After the nerve root is identified, retraction can be achieved with a retractor 288, or an instrument of the present invention can be used to precisely ablate at least a portion of the disc. If necessary, epidural veins are cauterized either automatically or with the coagulation mode of the present invention. If an annulotomy is necessary, it can be accomplished with a microknife or the ablation mechanism of the present invention while protecting the nerve root with the retractor 288. The herniated disc 290 is then removed with a pituitary rongeur in a standard fashion, or once again through ablation as described above.

In another embodiment, the present invention involves a channeling technique in which small holes or channels are formed within the disc 290, and thermal energy is applied to the tissue surface immediately surrounding these holes or channels to cause thermal damage to the tissue surface, thereby stiffening and debulking the surrounding tissue structure of the disc. Applicant has discovered that such stiffening of the tissue structure in the disc helps to reduce the pressure applied against the spinal nerves by the disc, thereby relieving back and neck pain.

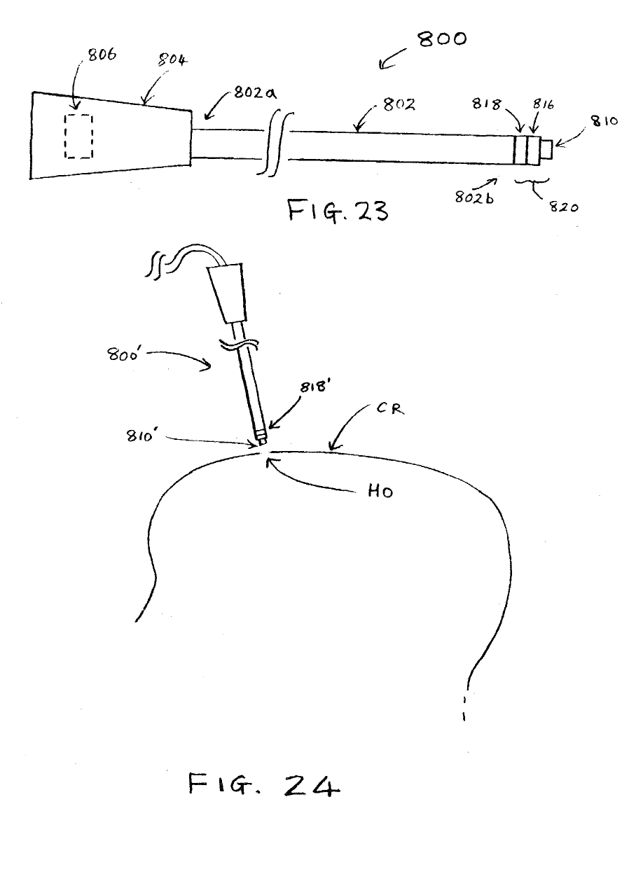

As shown in FIG. 21, the electrosurgical instrument 350 is introduced to the target site at the disc 290 as described above, or in another percutaneous manner (see FIGS. 23-25 below). The electrode assembly 351 is positioned adjacent to or against the disc surface, and electrically conductive fluid is delivered to the target site, as described above. Alternatively, the conductive fluid is applied to the target site, or the distal end of probe 350 is dipped into conductive fluid, e.g., liquid or gel, prior to introducing the probe 350 into the patient. The power supply 28 is then activated and adjusted such that a high frequency voltage difference is applied to the electrode assembly as described above.

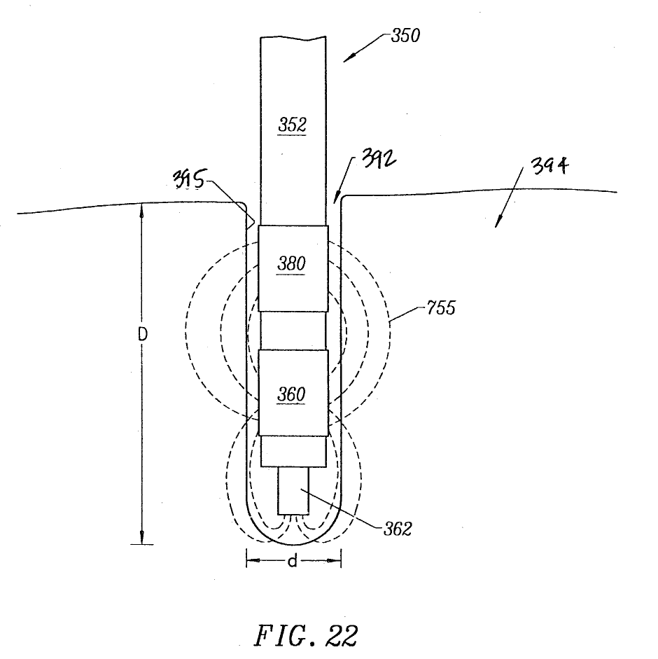

Depending on the procedure, the surgeon may translate or otherwise move the electrodes relative to the target disc tissue to form holes, channels, stripes, divots, craters or the like within the disc. In addition, the surgeon may purposely create some thermal damage within these holes, or channels to form scar tissue that will stiffen and debulk the disc. In one embodiment, the physician axially translates the electrode assembly 351 into the disc tissue as the tissue is volumetrically removed to form one or more holes 392 therein (see also FIG. 22). The holes 392 will typically have a diameter of less than 2 mm, preferably less than 1 mm. In another embodiment (not shown), the physician translates the active electrode across the outer surface of the disc to form one or more channels or troughs. Applicant has found that the present invention can quickly and cleanly create such holes, divots or channels in tissue with the cold ablation technology described herein. A more complete description of methods for forming holes or channels in tissue can be found in U.S. Pat. No. 5,683,366, the complete disclosure of which is incorporated herein by reference for all purposes.

FIG. 22 is a more detailed viewed of the probe 350 of FIG. 15D forming a hole 392 in a disc 290. Hole 392 is preferably formed with the methods described in detail above. Namely, a high frequency voltage difference is applied between active and return electrodes 362, 360, respectively, in the presence of an electrically conductive fluid such that an electric current 361 passes from the active electrode 362, through the conductive fluid, to the return electrode 360. As shown in FIG. 22, this will result in shallow or no current penetration into the disc tissue 394. The fluid may be delivered to the target site, applied directly to the target site, or the distal end of the probe may be dipped into the fluid prior to the procedure. The voltage is sufficient to vaporize the fluid around active electrode 362 to form a plasma with sufficient energy to effect molecular dissociation of the tissue. The distal end of the probe 350 is then axially advanced through the tissue as the tissue is removed by the plasma in front of the probe 350. The holes 392 will typically have a depth D in the range of about 0.5 cm to 2.5 cm, preferably about 1.2 cm to 1.8 cm, and a diameter d of about 0.5 mm to 5 mm, preferably about 1.0 mm to 3.0 mm. The exact diameter will, of course, depend on the diameter of the electrosurgical probe used for the procedure.

During the formation of each hole 392, the conductive fluid between active and return electrodes 362, 360 will generally minimize current flow into the surrounding tissue, thereby minimizing thermal damage to the tissue. Therefore, severed blood vessels on the surface 395 of the hole 392 may not be coagulated as the electrodes 362 advance through the tissue. In addition, in some procedures, it may be desired to thermally damage the surface 395 of the hole 392 to stiffen the tissue. For these reasons, it may be desired in some procedures to increase the thermal damage caused to the tissue surrounding hole 392. In the embodiment shown in FIG. 15D, it may be necessary to either: (1) withdraw the probe 350 slowly from hole 392 after coagulation electrode 380 has at least partially advanced past the outer surface of the disc tissue 394 into the hole 392 (as shown in FIG. 22); or (2) hold the probe 350 within the hole 392 for a period of time, e.g., on the order of 1 seconds to 30 seconds. Once the coagulation electrode is in contact with, or adjacent to, tissue, electric current 385 flows through the tissue surrounding hole 392 and creates thermal damage therein. The coagulation and return electrodes 380, 360 both have relatively large, smooth exposed surfaces to minimize high current densities at their surfaces, which minimizes damage to the surface 395 of hole. Meanwhile, the size and spacing of these electrodes 360, 380 allows for relatively deep current penetration into the tissue 394. In the representative embodiment, the thermal necrosis will extend about 1.0 mm to 5.0 mm from surface 395 of hole 392. In this embodiment, the probe may include one or more temperature sensors (not shown) on probe 350 coupled to one or more temperature displays on the power supply 28 such that the physician is aware of the temperature within the hole 392 during the procedure.

In other embodiments, the physician switches the electrosurgical system from the ablation mode to the sub-ablation or thermal heating mode after the hole 392 has been formed. This is typically accomplished by pressing a switch or foot pedal to reduce the voltage applied to a level below the threshold required for ablation for the particular electrode configuration and the conductive fluid being used in the procedure (as described above). In the sub-ablation mode, the physician will then remove the distal end of the probe 350 from the hole 392. As the probe is withdrawn, high frequency current flows from the active electrodes 362 through the surrounding tissue to the return electrode 360. This current flow heats the tissue and coagulates severed blood vessels at surface 395.

In another aspect of the invention, the size (e.g., diameter or principal dimension) of the active electrodes employed for treating the tissue are selected according to the intended depth of tissue treatment. As described previously in copending patent application PCT International Application, U.S. National Phase Serial No. PCT/US94/05168, the depth of current penetration into tissue increases with increasing dimensions of an individual active electrode (assuming other factors remain constant, such as the frequency of the electric current, the return electrode configuration, etc.). The depth of current penetration (which refers to the depth at which the current density is sufficient to effect a change in the tissue, such as collagen shrinkage, irreversible necrosis, etc.) is on the order of the active electrode diameter for the bipolar configuration of the present invention when operating at a frequency of about 100 kHz to about 200 kHz. Accordingly, for applications requiring a smaller depth of current penetration, one or more active electrodes of smaller dimensions would be selected. Conversely, for applications requiring a greater depth of current penetration, one or more active electrodes of larger dimensions would be selected.

FIG. 23 is a side view of an electrosurgical instrument 800 suitable for forming an access hole in a cranium of a patient, according to one aspect of the instant invention. Instrument or probe 800 generally includes a shaft 802 having a shaft proximal end 802a and a shaft distal end 802b. A handle 804 is affixed to shaft proximal end 802b. Handle 804 allows for manipulation of probe 800, and houses a connection block 806. An electrode assembly 820 is disposed at shaft distal end 802b. Electrode assembly 820 includes an active electrode 810 disposed on an electrically insulating electrode support or spacer 816, and a return electrode 818 spaced proximally from active electrode 810. The proximal spacing of return electrode 818 draws electrical current proximally from active electrode 810, thereby restricting the depth of penetration of current into the patient during use of probe 800. Active electrode 810 is schematically represented in FIG. 23, however, in practice, electrode 810 may have various configurations and geometries adapted for generating high current densities at one or more surfaces of electrode 810 (as described hereinabove) upon application of a high frequency voltage between electrode 810 and return electrode 818. Connection block 806 allows for the convenient, efficient, and facile electrical coupling of probe 800 to a high frequency power supply or electrosurgical generator (e.g., power supply 28, FIG. 1). In particular, active electrode 810 and return electrode 818 are independently coupled to the power supply via connection block 806.

FIG. 24 schematically represents formation of a hole, HO in a cranium, CR of a patient using an electrosurgical instrument 800′, according to one embodiment of the invention. Probe 800′ may include those structures, elements, and features described hereinabove for probe 800 (FIG. 23). After a suitable target location on the cranium has been selected, the target location may be prepared, e.g., in the conventional manner in preparation for forming a burr hole of the prior art. Thereafter, an active electrode 810′ may be positioned in at least close proximity to the cranium at the target location, and a high frequency voltage is applied between active electrode 810′ and a return electrode 818′. The high frequency voltage is applied from a high frequency power supply operating in the ablation mode. Typically, the high frequency voltage is within the range described hereinbelow (e.g., with reference to FIG. 31).

Prior to, or during application of the high frequency voltage, an electrically conductive fluid may be delivered to the distal end of probe 800′ to provide a current flow path between active electrode 810′ and return electrode 818′. The high frequency voltage is sufficient to effect volumetric removal (ablation) of bone tissue from the cranium at the target location. Typically, the ablation is effected via plasma-induced molecular dissociation of bone tissue components. Electrical current is drawn proximally by return electrode 818′, thereby restricting penetration of current into the patient. In this manner the depth of treatment can be precisely controlled, thereby avoiding damage to underlying or adjacent non-target tissue and preventing undue neuronal stimulation in the brain. In this manner, an access hole for performing various neurosurgical procedures may be provided electrosurgically, with less trauma to the patient as compared with using a mechanical burr. An access hole formed according to the invention typically has a diameter of about 14 mm or less, and more typically in the range of from about 2 mm to 8 mm. Although, the target location depicted in FIG. 24 is located just anterior to the coronal suture, apparatus and methods of the invention may also be used for forming an access hole at various other locations on the cranium.

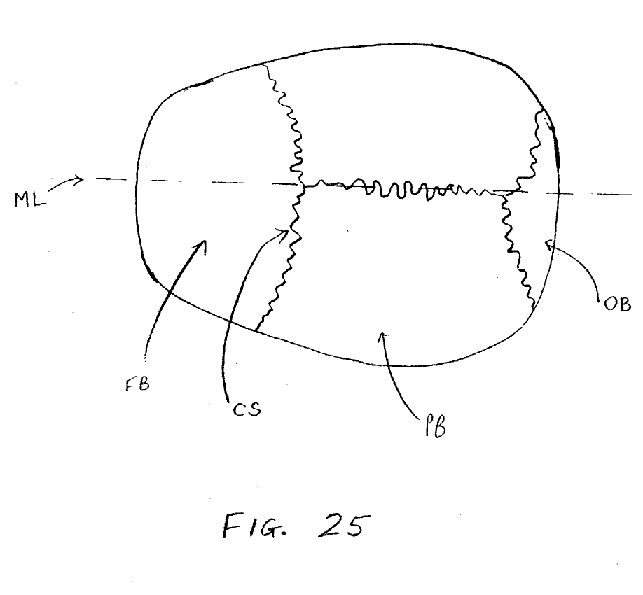

FIG. 25 is a superior view of the cranium showing the location of the midline, ML and coronal suture, CS in relation to the frontal bone, FB, the parietal bone, PB and the occipital bone, OB. In performing third ventriculostomy according to the invention, a typical target location for forming an access hole in the cranium is located up to about 5 cm anterior to the coronal suture, and from about 1 cm to 5 cm from the midline.

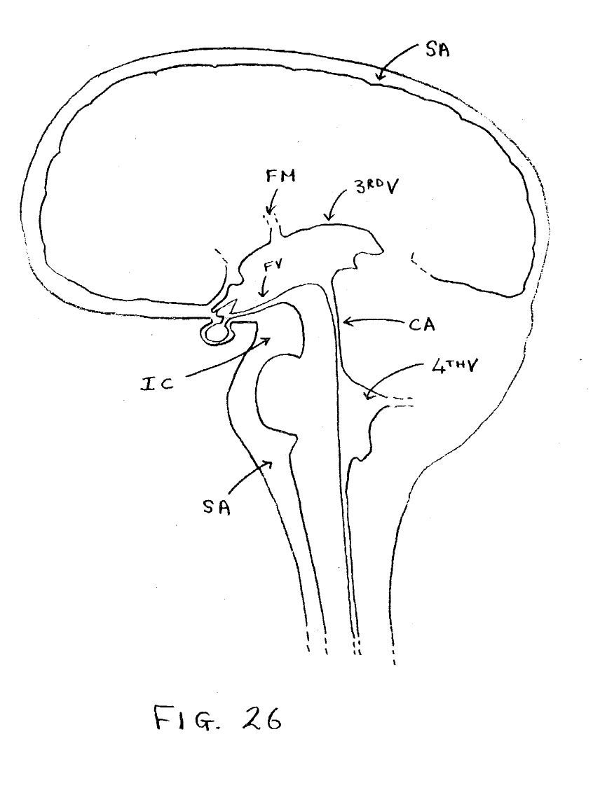

FIG. 26 shows the location of the third ventricle, 3RD V in relation to the interpeduncular cistern, IC of the sub-arachnoid space, SA. Also shown is the foramen of Monro, FM leading from the lateral ventricles (not shown) to the third ventricle, and the cerebral aqueduct, CA leading from the third ventricle to the fourth ventricle, 4^{TH} V. Circulation or flow of cerebrospinal fluid within the ventricles and to the sub-arachnoid space in a normal individual is known in the art. (See, for example, Plate 103 in F. H. Netter, M.D., Atlas of Human Anatomy, 2nd Edition, 3rd Printing, Novartis, East Hanover, N.J., 1999.) According to one aspect of the invention, excess accumulation of cerebrospinal fluid in the ventricles can be released to the sub-arachnoid space by electrosurgically fenestrating the boundary of the third ventricle (i.e., by forming a window, stoma, or drainage hole in the floor, FV of the third ventricle). In one embodiment, the floor of the third ventricle is fenestrated at a location inferior to the foramen of Monro and adjacent to the interpeduncular cistern. At this location, the boundary, or floor, of the third ventricle is relatively thin (as indicated in FIG. 26), and fenestration allows drainage of CSF to the interpeduncular cistern of the sub-arachnoid space.

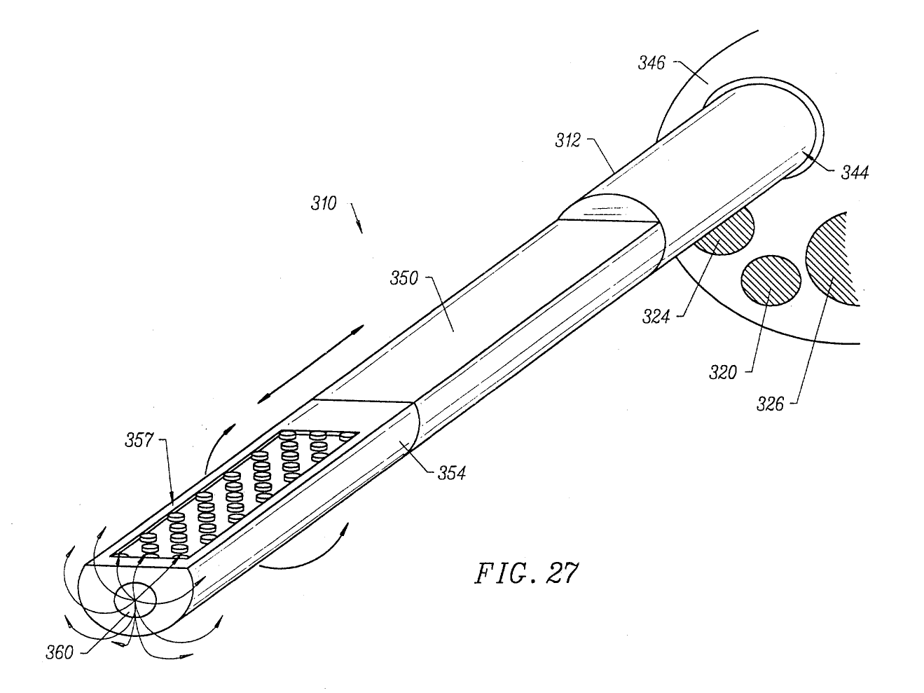

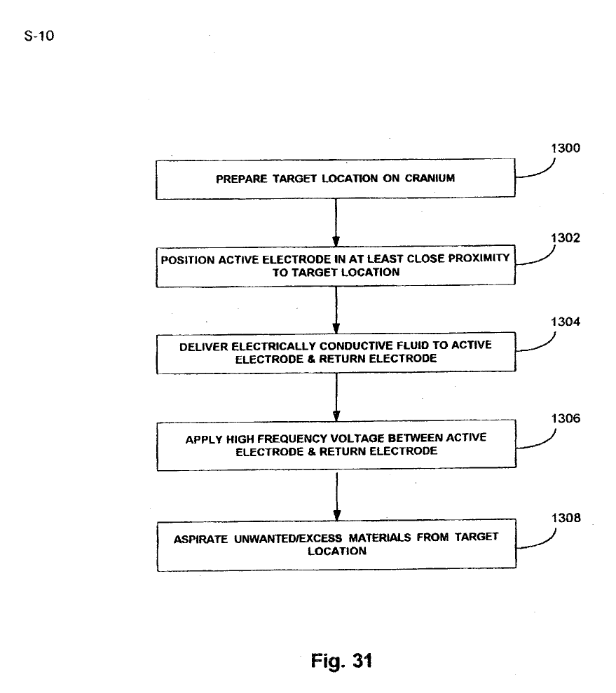

FIG. 27 is a side view of the distal end portion of an electrosurgical catheter 900, according to one embodiment of the invention. Catheter 900 includes an elongate flexible shaft 902, and an electrode assembly 920 disposed at a shaft distal or working end 902b. Typically, electrode assembly 920 includes a distal active electrode 910 and a return electrode 918 spaced proximally from active electrode 910 by an electrically insulating electrode support or spacer 916. Shaft 902 is adapted for being passed within a lumen of a cannula, endoscope, or the like. Typically, shaft 902 has a diameter in the range of from about 1 mm to 5 mm, and usually in the range of from about 1.5 mm to 3 mm. Shaft 902 is further adapted for being guided or steered within a patient such that shaft distal end 902b in general, and active electrode 910 in particular, is positioned in at least close proximity to a specific target location. Catheter 900 may further include a radiopaque tracking unit, located at shaft distal end 902b, for monitoring a location of shaft distal end 902b within the patient (e.g., FIG. 28A). In use, distal end 902b is usually positioned such that active electrode 910 is either in contact with a target tissue or within a few mm of the target tissue. By way of example, shaft 902 may be guided such that active electrode 910 is positioned in at least close proximity to the floor of the third ventricle at a location directly below the foramen of Monro (FIG. 26). According to another aspect of the invention, shaft 902 may be guided such that distal end 902b lies within the cerebral aqueduct adjacent to an occlusion within the cerebral aqueduct (FIG. 30). In one embodiment, active electrode 910 is adapted for both ablation of tissue (in the ablation mode) and for coagulation of tissue (in the sub-ablation mode). Alternatively, a separate coagulation electrode can be provided, generally as described hereinabove (e.g., with reference to FIGS. 15C, 15D, 17).

FIGS. 28A-D each show an electrosurgical instrument suitable for fenestrating the third ventricle of a hydrocephalus patient, according to four different embodiments of the invention. With reference to FIG. 28A, probe 1000 includes a shaft 1002 having a shaft proximal end 1002a and a shaft distal end 1002b. Shaft 1002 may comprise a plastic material or other non-metallic material that will allow visualization by magnetic resonance imaging (MRI) during a procedure. Typically, shaft 1002 has a diameter in the range of from about 1 mm to 5 mm, and usually in the range of from about 1.5 mm to 3 mm. Shaft 1002 typically has a length in the range of from about 5 cm to 30 cm, and usually in the range of from about 10 cm to 20 cm. An electrode assembly 1020 is disposed at shaft distal end 1002b. Electrode assembly 1020 includes a distal active electrode 1010 separated from a proximal return electrode 1018 by an electrically insulating electrode support or spacer 1016. Probe 1000 also includes a handle 1004 housing a connection block 1006. Probe 1000 further includes a mechanical stop 1034, a tracking unit 1030, and a plurality of depth markings 1032a-n.

As shown, mechanical stop 1034 is located at shaft proximal end 1002a. Mechanical stop 1034 limits the distance to which shaft distal end 1002b can be advanced through an introducer device (e.g., introducer 1040, FIG. 28B) by making mechanical contact with a proximal end of introducer 1040. Mechanical stop 1034 may be a rigid material or structure affixed to, or integral with, shaft 1002. Mechanical stop 1034 also serves to monitor the approximate depth or distance of advancement of shaft distal end 1002b through introducer 1040, and the degree of penetration of distal end 1002b into a patient's tissue, organ, or body. In one embodiment, mechanical stop 1034 is movable on shaft 1002, and stop 1034 includes a stop adjustment unit 1036 for adjusting the position of stop 1034 and for locking stop 1034 at a selected location on shaft 1002.

As shown, tracking unit 1030 is located at or near shaft distal end 1002b. In one embodiment, tracking unit 1030 includes a radiopaque material that can be visualized under fluoroscopy. Such a tracking unit 1030 provides the surgeon with visual input to track the position of shaft distal end 1002b relative to a specific target site to which active electrode 1010 is to be advanced. Such specific target sites may include, for example, an inner boundary or floor of the third ventricle (FIG. 26). Depth markings 1032a-n are distributed on shaft 1002, and serve to indicate to the surgeon the distance to which shaft distal end 1002b has been advanced into the patient, or the extent to which shaft 1002 has been introduced into introducer 1040. The surgeon can determine the position of active electrode 1010 by observing one or more of depth markings 1032a-n, or by comparing tracking unit output and a fluoroscopic image of the target site with a pre-operative fluoroscopic image of the target site.

With reference to FIG. 28B, probe 1000′ includes a proximal handle 1004′ and a shaft 1002′ having an electrode assembly 1020′ disposed at shaft distal end 1002′b. Electrode assembly 1020′ may have the same or analogous elements and configuration as electrode assembly 1020 (FIG. 28A). In addition, probe 1000′ may further include various other characteristics or elements described for probe 1000 with reference to FIG. 28A. Shaft 1002′ is adapted for being passed within a lumen of an introducer device 1040. As shown, introducer device 1040 includes a plurality of introducer markings 1042a-n for monitoring the extent of penetration of introducer 1040 into the patient.

With reference to FIG. 28C, probe 1000″ includes a shaft 1002″ having a shaft proximal end 1002″a and a shaft distal end 1002″b, a handle 1004″ at shaft proximal end 1002″a, and an electrode assembly 1020″ disposed at shaft distal end 1002″b. Electrode assembly 1020″ may be the same as, or analogous to, that described with reference to FIG. 28A. Shaft 1002″ is deflectable from a linear configuration, shown in dashed lines, to a curved configuration to allow electrode assembly 1020″ to be guided to a specific target location. In this way, shaft distal end 1002″b can be passed within a lumen of an introducer device (e.g., introducer 1040, FIG. 28B) in the linear configuration, and subsequently deflected after shaft distal end 1002″b has exited the lumen of the introducer device. Deflection of shaft distal end 1002″b and guiding of electrode assembly 1020″ to a target location can be achieved by use of pull wires, shape memory actuators, and the like. Probe 1000″ may further include various features and elements described for the embodiments depicted in FIGS. 28A-B.

FIG. 28D shows a probe 1000′″ including a shaft 1002′″ having a shaft proximal end 1002′″a and a shaft distal end 1002′″b, a handle 1004′″ at shaft proximal end 1002′″a, and an electrode assembly 1020′″ disposed at shaft distal end 1002′″b. Once again, electrode assembly 1020′″ may have the same or analogous elements as described for the embodiment of FIG. 28A. Probe 1000′″ may further include various features and elements described for the embodiments depicted in FIGS. 28A-C. Shaft 1002′″ includes a bend 1003′″, wherein shaft distal end 1002′″b lies at an angle ∝ in the range of from about 15° to 45° with respect to shaft proximal end 1002′″a. The presence of bend 1003 ″ in shaft 1002′″ enhances the surgeon's visibility of shaft distal end 1002′″b, e.g., during certain procedures performed under direct visualization.

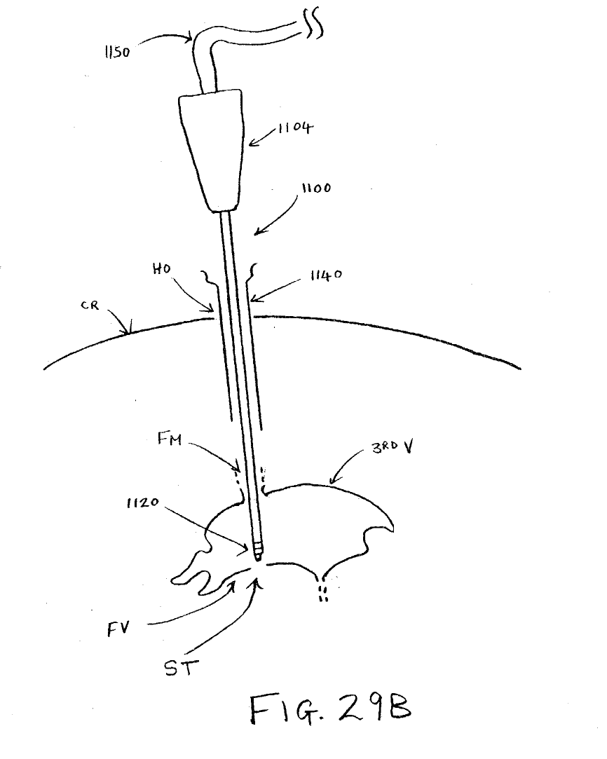

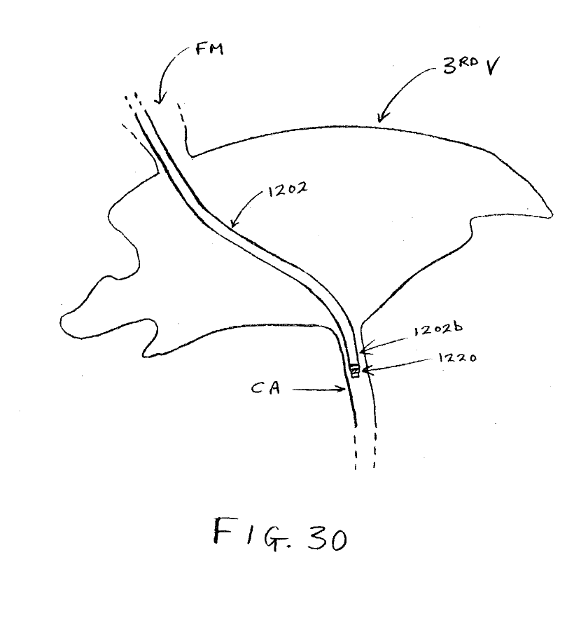

FIGS. 29A and 29B illustrate a method of fenestrating the third ventricle, according to one embodiment of the invention, in which an electrosurgical instrument or probe 1100 is advanced into the third ventricle, 3^{RD} V via a hole, HO in the patient's cranium, CR. In particular, a shaft 1102 is introduced through an introducer device 1140 such that the distal end of shaft 1102 enters the third ventricle via the foramen of Monro, FM, and wherein an electrode assembly 1120 is positioned in at least close proximity to a floor, FV of the third ventricle.

In one embodiment, electrode assembly 1120 has features and elements analogous to electrode assembly 1020 (FIG. 28A, supra), including a distal active electrode separated by a spacer from a proximal return electrode, wherein the active electrode may be adapted and configured for both ablating and coagulating tissue. The active and return electrodes are independently coupled to a high frequency power supply by a cable 1150. Cable 1150 is generally coupled to probe 1100 via a connection block (e.g., connection block 1006, FIG. 28A). Typically, the high frequency power supply is adapted for operation in either the ablation mode or the sub-ablation mode. In an alternative embodiment, electrode assembly 1120 includes a third, coagulation electrode (e.g., FIGS. 15C-D), wherein the coagulation electrode is configured for coagulating, shrinking, or stiffening tissue.

With reference to FIG. 29B, following the application of a suitable high frequency voltage to the active electrode, a stoma, ST is formed in the floor of the third ventricle, thereby allowing excess cerebrospinal fluid to drain from the third ventricle to the sub-arachnoid space (e.g., FIG. 26). Drainage of excess cerebrospinal fluid from the ventricles typically alleviates or eliminates the symptoms and sequelae of hydrocephalus. Optionally, the stoma can be enlarged by translating electrode assembly 1120 laterally with respect to the floor of the ventricle, e.g., by manipulating probe 1100 via a handle 1104.

FIG. 30 shows a shaft 1202 of an electrosurgical catheter introduced into the third ventricle, 3^{RD} V via the foramen of Monro, FM, for the treatment of obstructive hydrocephalus according to one embodiment of the invention. Typically, the catheter is passed within a cannula or endoscope (not shown), wherein the cannula or endoscope is introduced via an access hole in the cranium (e.g., FIG. 24). An electrode assembly 1220 is disposed at a distal or working end 1202b of shaft 1202. Electrode assembly 1220 typically includes a distal active electrode and a return electrode, and in some embodiments electrode assembly 1220 further includes a coagulation electrode (e.g., FIG. 17). Distal end 1202b is introduced into the cerebral aqueduct, CA by steering or guiding shaft 1202, e.g., under fluoroscopy. As an example, shaft 1202 may be guided via use of pull wires, or shape memory actuators. In normal individuals, the cerebral aqueduct allows CSF to flow from the third ventricle to the fourth ventricle (FIG. 26), and thence to the sub-arachnoid space. Obstruction of the cerebral aqueduct, known as aqueductal stenosis, is the most common cause of congenital hydrocephalus.

Again with reference to FIG. 30, electrode assembly 1220 is advanced within the endoscope until electrode assembly 1220 lies distally to the distal end of the endoscope, and the active electrode is positioned in at least close proximity to a blockage or occlusion of the cerebral aqueduct. Thereafter, a first high frequency voltage is applied between the active and return electrodes of electrode assembly 1220 from a power supply (e.g., power supply 28, FIG. 1), wherein the power supply is operating in the ablation mode, and the first high frequency voltage is sufficient to volumetric remove occluding material within the cerebral aqueduct. In this manner a channel is provided through the occluding material, whereby patency of the cerebral aqueduct is established (in the case of congenital stenosis) or re-established (e.g., in tumor-related stenosis). Typically, the first high frequency voltage is in the range of from about 50 volts RMS to 1000 volts RMS, and more typically from about 100 volts RMS to 500 volts RMS. Optionally, tissue adjacent to the channel may be coagulated to provide stiffening or firming of tissue lying adjacent to the channel. Applicant believes that such stiffening or firming of tissue serves to maintain patency of the channel formed in the cerebral aqueduct according to the invention. Coagulation of tissue may be achieved by applying a second, usually lower, voltage, either to the active electrode or to a coagulation electrode (e.g., FIG. 17). Typically, the second high frequency voltage is in the range of from about 10 volts RMS to 500 volts RMS, and more typically from about 20 volts RMS to 200 volts RMS.

FIG. 31 schematically represents a series of steps involved in a method of forming an access hole in the cranium of a patient using an electrosurgical instrument or probe, according to one embodiment of the present invention. In general, forming an access hole in the cranium electrosurgically provides an alternative to using a mechanical burr of prior art methods. (In the prior art, burr holes are commonly formed to provide access to the brain in various conventional neurosurgical procedures.) Again with reference to FIG. 31, step 1300 involves preparing a target location on the cranium. In one embodiment of the instant invention, the access hole may be provided to gain access to the third ventricle in a ventriculostomy procedure, and the target location is selected in a region of the cranium up to about 5 cm anterior to the coronal suture and from about 1 cm to 5 cm from the midline. In one embodiment, an electrically conductive fluid, e.g., in the form of a viscous gel, may be applied to the scalp at the target location.

Step 1302 involves positioning at least one active electrode of the electrosurgical probe in at least close proximity to the cranium at the target location. The probe includes a return electrode, usually located at the distal end of the probe shaft proximal to the active electrode(s). In one embodiment, a single active electrode is located at the distal terminus of the probe shaft. Alternatively, the probe may have an array of active electrodes disposed on an electrically insulating support. The active electrode(s) have a geometry which promotes high current density at one or more surfaces of the active electrode(s).

Typically, the probe further includes a fluid delivery element or unit (e.g., FIGS. 8B, 8C). The fluid delivery unit is adapted for delivering an electrically conductive fluid to the distal end of the probe shaft. Step 1304 involves delivering electrically conductive fluid to the distal end of the probe shaft, e.g., via the fluid delivery unit, so as to provide a current flow path between the active and return electrodes. The electrically conductive fluid may be isotonic saline delivered from a fluid source. Alternatively, an electrically conductive gel may be applied to the target location or to the distal end of the probe.

Step 1306 involves applying a high frequency voltage, from a high frequency power supply or generator, between the active and return electrodes, wherein the power supply is operating in the ablation mode. The high frequency voltage applied in step 1306 is sufficient to volumetrically remove tissue of the cranium at the target location, whereby an access hole is formed in the cranium. Such an access hole is functionally analogous to a burr hole of the prior art. The access hole formed according to the invention may have a diameter up to about 14 mm, and more usually in the range of from about 2 mm to 8 mm. Typically, the voltage applied in step 1306 is in the range of from about 100 volts RMS to 1800 volts RMS, usually 200 volts RMS to 1500 volts RMS, and often 300 volts RMS to 1200 volts RMS. The applied voltage is generally sufficient to vaporize the electrically conductive fluid and to ionize the vapor in the vicinity of the active electrode to generate a plasma. In an exemplary embodiment, removal of cranium tissue is effected via plasma-induced molecular dissociation of cranium tissue components. During step 1306 fragments of cranium tissue and excess electrically conductive fluid may tend to accumulate in the region of the target location. Step 1308 involves aspirating these and any other excess or unwanted materials from the surgical site. In one embodiment, the probe includes an integral aspiration or suction unit adapted for the removal of such excess or unwanted materials from the surgical site (e.g., FIGS. 4, 5).

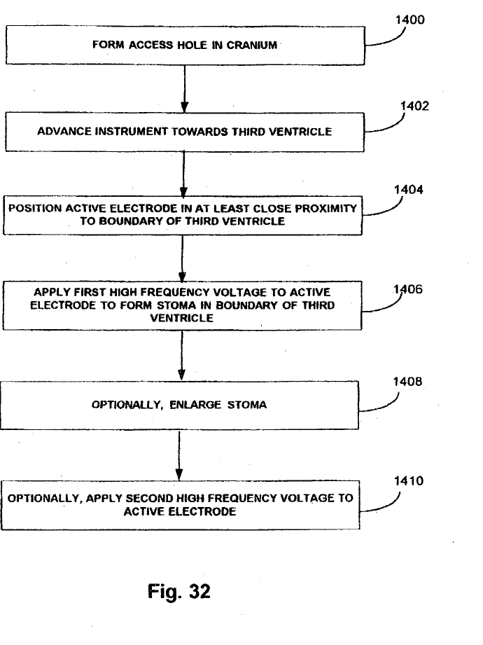

FIG. 32 schematically represents a series of steps involved in a method of performing a third ventriculostomy, according to one embodiment of the invention, wherein step 1400 involves forming an access hole at a target location in the cranium of a patient. The access hole may be formed mechanically, e.g., using a burr. Alternatively, the access hole may be formed using an electrosurgical instrument, e.g., as described with reference to FIG. 31, supra. Typically, the access hole has a diameter in the range of from about 2 mm to 8 mm. In one embodiment, the access hole is formed at a location somewhat anterior to the coronal suture, for example, up to about 5 cm anterior to the coronal suture, and in the range of from about 1 cm to 5 cm from the midline. An access hole formed in this region of the cranium enables substantially direct access to the floor of the third ventricle via the foramen of Monro.

Step 1402 involves advancing an electrosurgical instrument, via the access hole, towards the third ventricle. The electrosurgical instrument may include those elements and characteristics described hereinabove, e.g., with reference to FIGS. 1-15, 17, 27, 28A-D, and 30). For example, the instrument may be an electrosurgical probe having an elongate shaft, wherein the shaft may be substantially linear (e.g., FIGS. 28A-C) or bent (e.g., FIG. 28D). The distal end of the probe shaft may be deflectable (e.g., FIG. 28C) upon application of a suitable force to the shaft (e.g., via pull wires), so as to allow guiding of the shaft distal end to a specific target location of the patient. Such a probe may be advanced towards the third ventricle through a lumen of an introducer device (e.g., FIGS. 28B, 29A-B). Alternatively, the instrument may be an electrosurgical catheter including a flexible shaft, having a steerable shaft distal end. Such an electrosurgical catheter may be adapted for passage within a flexible or rigid endoscope.

The electrosurgical instrument (probe or catheter) includes an electrode assembly disposed at the shaft distal end. In one embodiment, the electrode assembly includes an active electrode spaced from a return electrode, wherein the active electrode is adapted for ablating tissue. The active electrode may also be adapted for coagulating tissue and for effecting hemostasis at the surgical site. In an alternative embodiment, the electrode assembly includes a separate coagulation electrode, in addition to an active electrode and a return electrode (e.g., FIGS. 15C-D, 17). Step 1404 involves positioning the active electrode of the instrument in at least close proximity to a boundary of the third ventricle. In one embodiment, the distal or working end of the instrument lies within the third ventricle such that the active electrode is positioned in at least close proximity to the floor of the third ventricle, in the region marked FV in FIG. 26.

Step 1406 involves applying a first high frequency voltage between the active electrode and the return electrode, wherein the first high frequency voltage is sufficient to locally ablate the boundary of the third ventricle so as to form a stoma, window, or drainage hole in the floor of the third ventricle. Such a stoma in the floor of the third ventricle allows excess cerebrospinal fluid to drain from the ventricles into the interpeduncular cistern of the sub-arachnoid space. Such drainage of cerebrospinal fluid typically alleviates or eliminates symptoms of hydrocephalus. The first high frequency voltage is typically in the range of from about 50 volts RMS to 1000 volts RMS, and usually from about 100 volts RMS to 500 volts RMS. In contrast to prior art ventriculostomies which employ mechanical devices, such as closed forceps, to puncture the floor of the third ventricle, often resulting in excessive bleeding, methods and apparatus of the invention allow for simultaneous hemostasis and ablation at the target site. Any residual bleeding may be arrested by application of a second, lower voltage to the electrode assembly (as described for step 1410, infra).

Optional step 1408 involves enlarging the stoma. For example, the stoma can be enlarged by translating the active electrode laterally with respect to the floor of the third ventricle while continuing to apply the first high frequency voltage. Alternatively, the stoma may be enlarged mechanically, for example, using a balloon catheter (the latter well known in the art). After formation of a stoma of suitable dimensions, optional step 1410 involves applying a second high frequency voltage to the electrode assembly, wherein the second high frequency voltage is selected to coagulate tissue adjacent to the stoma in the third ventricle. Application of the second high frequency voltage serves to effect hemostasis in cases where there is residual bleeding after completion of step 1406. In addition, application of the second high frequency voltage stiffens the tissue adjacent to the stoma, thereby helping to maintain patency of the stoma.

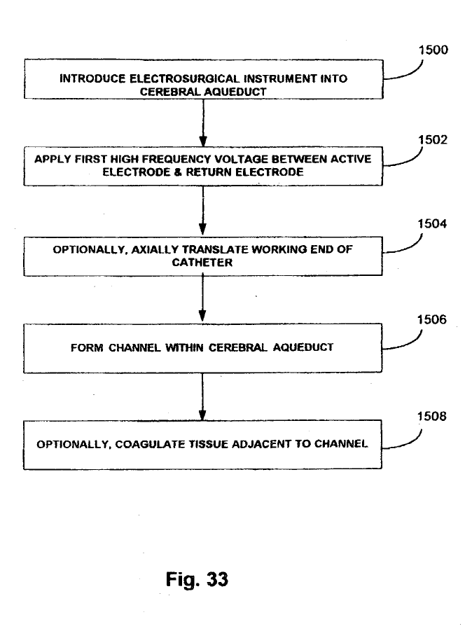

FIG. 33 schematically represents a series of steps involved in a method of establishing patency in the cerebral aqueduct of a patient, according to another embodiment of the invention. The method is particularly applicable to patient's having obstructive hydrocephalus due to aqueductal stenosis. Such aqueductal stenosis may be congenital or tumor-related. Step 1500 involves introducing an electrosurgical instrument into the cerebral aqueduct of the patient. The instrument may be an electrosurgical catheter including a steerable shaft having an electrode assembly disposed at the shaft distal or working end. The catheter may have features, characteristics, or elements described hereinabove, e.g., with reference to FIGS. 17, 27, 29A-B. The electrode assembly includes an active electrode and a return electrode spaced proximally from the active electrode. The active electrode is adapted for volumetric removal of tissue upon application of a high frequency voltage between the active and return electrodes. In one embodiment, step 1500 involves passing the shaft of the catheter within an endoscope, such that the shaft enters the third ventricle via the foramen of Monro. The shaft distal end is then guided into the cerebral aqueduct such that the active electrode is positioned in at least close proximity to a target tissue. Typically, the target tissue comprises an occlusion within the cerebral aqueduct. Guiding the shaft distal end may be performed during visualization of the location of the shaft relative to the target tissue, for example the visualization may be performed endoscopically or via fluoroscopy.

Step 1502 involves applying a first high frequency voltage between the active electrode and the return electrode, wherein the first high frequency voltage is sufficient to volumetrically remove at least a portion of the occlusion within the cerebral aqueduct. The first high frequency voltage applied in step 1502 is generally within the range cited hereinabove for ablation of tissue, for example, as recited for step 1406 (FIG. 32). When appropriate, the distal end of the catheter shaft may be axially translated during application of the first high frequency voltage, according to optional step 1504. As a result, a channel is formed through the occlusion in the cerebral aqueduct (step 1506), whereby excess cerebrospinal fluid can drain from the third ventricle to the fourth ventricle, and thence to the sub-arachnoid space.

Optional step 1508 involves coagulating tissue adjacent to the channel within the cerebral aqueduct. Typically, the tissue is coagulated by applying a second high frequency voltage to the electrode assembly from a high frequency power supply, wherein the power supply is operating in the sub-ablation or coagulation mode. The second high frequency voltage is generally within the range stated hereinabove for the sub-ablation mode, e.g., as described with reference to step 1410 (FIG. 32). In one embodiment, the second high frequency voltage serves to stiffen the tissue adjacent to the channel within the cerebral aqueduct, whereby patency of the cerebral aqueduct is maintained.

While the exemplary embodiments of the present invention have been described in detail, by way of example and for clarity of understanding, a variety of changes, adaptations, and modifications will be obvious to those of skill in the art. Therefore, the scope of the present invention is limited solely by the appended claims.

What is claimed is:

1. A method of fenestrating a third ventricle of a patient, comprising:
a) positioning a distal end of an electrosurgical instrument in at least close proximity to a boundary of a third ventricle of the patient, the instrument having an active electrode and a return electrode, the return electrode spaced from the active electrode; and
b) applying a first high frequency voltage between the active electrode and the return electrode, wherein the first high frequency voltage is sufficient to form a stoma in the boundary of the third ventricle.

2. The method of claim 1, wherein said step a) comprises positioning the active electrode in at least close proximity to an inner boundary of the third ventricle.

3. The method of claim 1, wherein the first high frequency voltage is sufficient to ablate the boundary of the third ventricle in the vicinity of the active electrode.

4. The method of claim 1, wherein the first high frequency voltage simultaneously effects both ablation of a target tissue and hemostasis adjacent to the target tissue.

5. The method of claim 4, wherein the target tissue comprises the floor of the third ventricle.

6. The method of claim 1, wherein said step b) comprises applying the first high frequency voltage in the range of from about 100 volts RMS to 500 volts RMS.

7. The method of claim 1, wherein the stoma is formed by localized volumetric removal of the floor of the third ventricle.

8. The method of claim 1, wherein the stoma allows cerebrospinal fluid to flow from the third ventricle to the interpeduncular cistern.

9. The method of claim 1, further comprising enlarging the stoma.

10. The method of claim 9, wherein the stoma is enlarged electrosurgically by the localized volumetric removal of the boundary of the third ventricle.

11. The method of claim 9, wherein said step c) comprises: while applying the first high frequency voltage of said step b), manipulating the instrument such that the active electrode is translated laterally with respect to the stoma.

12. The method of claim 9, wherein the stoma is enlarged by placement of a balloon catheter in the stoma.

13. The method of claim 1, wherein the active electrode and the return electrode are independently coupled to a high frequency power supply.

14. The method of claim 13, wherein said step b) comprises applying the first high frequency voltage in an ablation mode, and the method further comprises:
d) after said step b), switching the high frequency power supply to a sub-ablation mode; and
e) thereafter, applying a second high frequency voltage between the active electrode and the return electrode, wherein the second high frequency voltage is sufficient to coagulate tissue adjacent to the stoma.

15. The method of claim 14, wherein said step e) effects hemostasis in the vicinity of the active electrode.

16. The method of claim 14, wherein the second high frequency voltage is in the range of from about 20 volts RMS to 200 volts RMS.

17. The method of claim 1, wherein said step a) comprises introducing a distal end of an endoscope into the third ventricle of the patient.

18. The method of claim 17, wherein the endoscope is introduced into the third ventricle via a foramen of Monro.

19. The method of claim 17, wherein the electrosurgical instrument comprises a catheter, the active electrode disposed at a distal end of the catheter, and the method further comprises:
f) advancing the catheter through the endoscope such that the distal end of the catheter is located within the third ventricle.

20. The method of claim 17, wherein the endoscope is flexible.

21. The method of claim 1, wherein the electrosurgical instrument comprises an electrosurgical probe including a shaft having a shaft distal end, the active electrode disposed at the shaft distal end, and the method further comprises:
g) advancing the shaft distal end through a lumen of a rigid introducer device such that the shaft distal end is located within the third ventricle.

22. A method of performing a third ventriculostomy on a patient, comprising:
a) forming an access hole at a target location in the cranium of the patient;
b) via the access hole, advancing an electrosurgical instrument towards the third ventricle of the patient;
c) positioning an active electrode of the instrument in at least close proximity to a boundary of the third ventricle, the instrument having a return electrode spaced from the active electrode; and
d) applying a first high frequency voltage between the active electrode and the return electrode, wherein the first high frequency voltage is sufficient to form a stoma in the boundary of the third ventricle.

23. The method of claim 22, wherein the target location of said step a) is located up to about 5 cm anterior to the coronal suture and in the range of from about 1 cm to 5 cm from the midline.

24. The method of claim 22, wherein said step a) comprises forming the access hole electrosurgically via plasma-induced volumetric removal of cranium tissue at the target location.

25. The method of claim 22, wherein the access hole comprises a burr hole formed mechanically by a burr.

26. The method of claim 22, wherein the instrument comprises an electrosurgical catheter, and the method further comprises:
e) prior to said step b), introducing an endoscope into the third ventricle.

27. The method of claim 26, wherein said step b) comprises advancing the catheter within the endoscope such that a distal end of the catheter is located within the third ventricle, and wherein the active electrode is disposed on the distal end of the catheter.

28. The method of claim 26, wherein the endoscope is flexible or rigid.

29. The method of claim 22, wherein the instrument comprises an electrosurgical probe, and the method further comprises:
f) prior to said step b), introducing an introducer device into the patient via the access hole.

30. The method of claim 29, wherein the introducer device comprises a rigid tube having a lumen therethrough.

31. The method of claim 30, wherein said step b) comprises passing a distal end of the probe through the lumen of the introducer device.

32. The method of claim 22, wherein the first high frequency voltage is sufficient to effect localized ablation of the boundary of the third ventricle in the vicinity of the active electrode.

33. The method of claim 22, further comprising:
g) enlarging the stoma to form a channel through the boundary of the third ventricle.

34. The method of claim 33, wherein said step g) comprises enlarging the stoma by translating the active electrode laterally with respect to the stoma.

35. The method of claim 22, wherein the active electrode and the return electrode are independently coupled to a high frequency power supply, the high frequency power supply adapted for operating in an ablation mode or a sub-ablation mode.

36. The method of claim 22, further comprising:
h) applying a second high frequency voltage between the active electrode and the return electrode, wherein the second high frequency voltage is sufficient to effect hemostasis in the vicinity of the active electrode.

37. The method of claim 36, wherein the first high frequency voltage is in the range of from about 100 volts RMS to 500 volts RMS, and the second high frequency voltage is in the range of from about 20 volts RMS to 200 volts RMS.

38. The method of claim 36, wherein the second high frequency voltage serves to stiffen tissue adjacent to the stoma.

39. The method of claim 22, wherein said step c) comprises positioning the active electrode in at least close proximity to a floor of the third ventricle.

40. The method of claim 22, wherein the stoma in the boundary of the third ventricle allows excess cerebrospinal fluid to drain from the third ventricle, and symptoms of obstructive hydrocephalus are alleviated.

41. The method of claim 22, wherein the instrument includes at least one depth marking, and said step b) comprises monitoring a location of the at least one depth marking with respect to the cranium at the target location.

42. The method of claim 22, wherein at least one of said step b) or said step c) is performed under fluoroscopy.

43. The method of claim 22, wherein the instrument includes a tracking unit, the tracking unit disposed at a shaft distal end of the instrument, and the method further comprises:
after said step a), monitoring a location of the shaft distal end of the instrument within the patient.

44. The method of claim 22, wherein the instrument further includes a coagulation electrode adapted for coagulating tissue and for effecting hemostasis.

45. A method of forming an access hole in the cranium of a patient using an electrosurgical probe, comprising:
a) positioning an active electrode of the probe in at least close proximity to a target location of the cranium;
b) delivering an electrically conductive fluid to the active electrode or to the target location; and
c) applying a high frequency voltage between the active electrode and a return electrode, wherein the high frequency voltage is sufficient to locally ablate tissue at the target location, whereby the access hole is formed in the cranium at the target location.

46. The method of claim 45, wherein the electrically conductive fluid provides a current flow path between the active electrode and the return electrode.

47. The method of claim 45, wherein the high frequency voltage is sufficient to effect the controlled removal of cranium tissue at the target location.

48. The method of claim 45, wherein the access hole has a diameter in the range of from about 2 mm to 8 mm.

49. The method of claim 45, wherein the high frequency voltage applied in said step c) is in the range of from about 100 volts RMS to 1800 volts RMS.

50. The method of claim 45, wherein the probe includes a fluid delivery element for delivering the electrically conductive fluid to the distal end of the probe or to the target location.

51. The method of claim 45, wherein said step b) comprises placing an electrically conductive gel on the scalp of the patient at the target location.

52. The method of claim 45, wherein the probe includes an aspiration unit, and the method further comprises:
d) aspirating tissue fragments and excess electrically conductive fluid from the target location.

53. A method of establishing patency in a cerebral aqueduct of a hydrocephalus patient, comprising:
a) introducing a distal end of an electrosurgical catheter into the cerebral aqueduct of the patient, wherein the catheter includes an electrode assembly disposed on the distal end, the electrode assembly including an active electrode and a return electrode; and
b) applying a first high frequency voltage between the active electrode and the return electrode, the first high frequency voltage sufficient to ablate tissue, wherein tissue adjacent to the active electrode is volumetrically removed and patency of the cerebral aqueduct is established.

54. The method of claim 53, wherein the first high frequency voltage is in the range of from about 100 volts RMS to 500 volts RMS.

55. The method of claim 53, wherein said step a) comprises positioning the active electrode in at least close proximity to a target tissue.

56. The method of claim 55, wherein the target tissue comprises an occlusion of the cerebral aqueduct.

57. The method of claim 53, further comprising:
c) during said step b), axially translating the active electrode.

58. The method of claim 55, wherein the target tissue occludes the cerebral aqueduct, and wherein volumetric removal of the target tissue forms a channel between a third ventricle and a fourth ventricle of the patient.

59. The method of claim 58, further comprising:
d) while the electrode assembly is positioned within the channel, applying a second high frequency voltage between the active electrode and the return electrode, the second high frequency voltage selected to coagulate tissue adjacent to the channel.

60. The method of claim 59, wherein the second high frequency voltage is in the range of from about 20 volts RMS to 200 volts RMS.

61. The method of claim 59, wherein hemostasis within the cerebral aqueduct is effected by said step b) or by said step d).

62. The method of claim 59, wherein symptoms associated with aqueductal stenosis are alleviated or eliminated.

63. An apparatus for performing electrosurgical third ventriculostomy on a hydrocephalus patient, the apparatus comprising:
a shaft having a shaft distal end and a shaft proximal end, the shaft having at least one depth marking thereon, the shaft distal end adapted for passage into a third ventricle of the patient via a foramen of Monro, and the shaft having a diameter in the range of from about 1 mm to 5 mm;
an electrode assembly disposed at the shaft distal end, the electrode assembly including a distal active electrode and a proximal return electrode, the return electrode spaced from the active electrode by an electrically insulating spacer; and
a tracking unit disposed at the shaft distal end, the tracking unit adapted for monitoring a location of the shaft distal end with respect to the third ventricle of the patient.

64. The apparatus of claim 63, further comprising an introducer device adapted for insertion in an access hole in the cranium of the patient, the introducer device having an introducer lumen therethrough, the introducer lumen adapted for passage of the shaft distal end therethrough.

65. The apparatus of claim 63, wherein the electrode assembly further includes a coagulation electrode.

66. The apparatus of claim 63, further comprising a high frequency power supply, the active electrode and the return electrode independently coupled to the high frequency power supply via a connection block.

67. The apparatus of claim 66, further comprising a voltage reduction element coupled between the power supply and the active electrode.

68. The apparatus of claim 63, wherein at least the shaft distal end is steerable to a specific target site within the patient.

69. The apparatus of claim 63, wherein the shaft consists essentially of a nonmetallic material.

70. The apparatus of claim 63, wherein the shaft has a bend at an angle in the range of from about 15° to 45°.

71. The apparatus of claim 63, wherein the tracking unit comprises a radiopaque material.
